(19) United States
(12) Patent Application Publication
(10) Pub. No.: US 2024/0026434 A1
(43) Pub. Date: Jan. 25, 2024

(54) LYOPHILIZED COMPOSITIONS, MATERIALS, AND METHODS

(71) Applicant: New England Biolabs Lyophilization Sciences Ltd., Hitchin (GB)

(72) Inventors: Martin Alan Lee, Salisbury (GB); James Slatter, Salisbury (GB)

(73) Assignee: New England Biolabs Lyophilization Sciences Ltd., Hitchin (GB)

(21) Appl. No.: 18/355,890

(22) Filed: Jul. 20, 2023

Related U.S. Application Data
(60) Provisional application No. 63/369,136, filed on Jul. 22, 2022.

(51) Int. Cl. C12Q 1/6851; C12Q 1/6806; B01L 3/00

(52) U.S. Cl. CPC C12Q 1/6851; C12Q 1/6806; B01L 3/50825; B01L 2200/16; B01L 2300/0832

(57) ABSTRACT

There is provided a method of preparing a lyophilized sample, comprising: (i) adding a sample comprising one or more molecules of interest in solution to a porous polymer matrix and allowing the sample to penetrate into the pores of the porous polymer matrix; and (ii) lyophilizing the sample within the pores of the porous polymer matrix thereby preparing a porous polymer matrix containing the lyophilized sample.

### BACKGROUND

Lyophilization is the creation of a stable preparation of a substance by rapid freezing and dehydration of the frozen product under high vacuum. Under controlled temperature and pressure, water sublimes moving from the solid into the vapor state. A condenser then drives diffusion to remove water vapor from the substance. Using carefully controlled changes in temperature and pressure, >95% of the original water content may be removed from the substance. In the presence of protectants, many biological materials may be frozen in a manner that leaves them both intact and active. Freeze-drying protectants include sugars that are naturally found in organisms that can withstand environmentally harsh conditions such as low temperature and drought. Along with a combination of anti-oxidizing agents and bulking materials they form the “excipient” or “carrier” material for the dried biologic. They are usually the largest material by mass in the lyophilized product and form a structured “cake”, retaining the dried biologic ingredient in a stabilized state. Various improvements in the freeze-drying apparatus and methods have been described in US 2020/0299789, WO 2013/171483, WO 2021/105284.

Lyophilization has been applied to a wide variety of molecular biological reagent types, including complex mixtures suitable for rapid dissolution for use in laboratory process automation (see for example U.S. Pat. No. 10,626,472, WO 2021/105284). Lyophilization of reagents also provides long shelf-life at ambient temperature which is desirable for warehousing, transport and end-user storage of the product.

Although the conditions for lyophilizing products are well established, some improvements are desirable. For example, ovens are used to remove water vapour from the frozen products. The ovens have limited capacity for large batches of product. It would be desirable to increase the capacity of existing ovens for large numbers of samples. To this end, certain vendors have developed lyobeads (see for example, Millrock Technology (Kingston, NY) LyoBeads) also U.S. Pat. No. 11,197,905 in which the products to be dried in the oven are in bead shaped cakes that can be dried on trays.

Additionally, the size and shape of a stabilized agent is largely dictated by the size and shape of its container during freeze-drying. For example, material dried in a cylindrical vial will form a cylindrical cake in the bottom of the vial, occupying the same space as the liquid starting material. Freeze-drying process considerations, options for robotic handling, and other factors limit usable container shapes and sizes and frequently require customization of the lyophilized product that is time consuming and costly. Moreover, lyophilized reagents in the form of a cake are not always suited for efficient handling in automated workflows, diagnostic devices or therapeutic treatments.

More specifically there is an increasing desire for the manufacture of high volume diagnostic point of care test devices for use, for example, in detecting pathogens where such tests are stable at ambient temperatures. High volume drying is important for these processes as they are carried out in batches that involve high cost capital equipment and fixed operational costs. Here too there is a need for flexibility in shapes, sizes, and/or quantities of stabilized reagents for introduction to various reaction vessels involving different devices.

### SUMMARY

Accordingly, needs have arisen for improved systems, apparatus, compositions, kits, workflows, and/or methods that provide one or more dried reagents in one or more desired forms. The present disclosure relates to such systems, apparatus, compositions, kits, workflows, and methods. For example, a method of producing a lyophilized sample may comprise lyophilizing a material comprising a porous polymer matrix and a sample solution in pores of the matrix, the sample solution comprising one or more molecules of interest, thereby producing a porous polymer matrix comprising the lyophilized sample. According to some embodiments, a method may further comprise contacting the sample solution and the porous polymer matrix to form the material that is to be lyophilized. A porous polymer matrix (e.g., a porous polymer matrix contacted with a sample solution) may have any desired shape or form including, for example, the form of a film, a sheet, a strip, a tape, a block, a cylinder, or a bead. In some embodiments, a method may include forming (e.g., by cutting, stamping, punching, tearing, or fragmenting) the porous polymer matrix comprising the lyophilized sample into one or more pieces having a desired mass, size, volume, and/or shape. A method may further include, according to some embodiments, inserting the piece(s) of lyophilized polymer matrix into one or more reaction volumes (e.g., in a reaction container and/or test device); optionally wherein the piece(s) of polymer matrix comprises a quantity of the one or more molecules of interest that is effective for a reaction in the reaction volume(s). To arrive at an effective amount, a user may take into consideration one or more factors including the activity of the molecule of interest, its role in the reaction(s) to be performed (e.g. reactant, substrate, catalyst, cofactor), concentration in a liquid, prior to lyophilization, reconstitution conditions (e.g., volume, efficiency). A sample solution, for example, a sample solution for lyophilization in a porous polymer matrix, may include a defined concentration or a predetermined amount of the one or more molecules of interest. In some embodiments, the defined concentration or a predetermined amount of the one or more molecules of interest may be proportioned to produce a defined or predetermined mass to mass or mass to volume ratio of (a)(i) lyophilized sample or (ii) molecule(s) of interest to (b) porous polymer matrix. A method may further include storing the porous polymer matrix containing the lyophilized sample. A stored polymer matrix comprising lyophilized sample may have any desired form, for example, one or more flat sheets or strips (e.g., layered flat sheets or strips) or a tape (e.g., a pliable tape) arranged as a roll (e.g., wrapped around a spindle or cassette). A method, in some embodiments, may comprise, after the lyophilizing step, sealing the porous polymer matrix containing the lyophilized sample, for example, by reversibly bonding the porous polymer matrix to an impermeable membrane or reversibly encapsulating the porous polymer matrix within an impermeable material.

The present disclosure also provides lyophilized materials including, for example, lyophilized samples comprising one or more molecules of interest, the lyophilized samples occupying or otherwise contained in pores of one or more porous polymer matrices. A porous polymer matrix may have any desired shape or form including, for example, the form of a film, a sheet, a strip, a tape, a block, a cylinder, or a bead. In some embodiments, a lyophilized sample contained in pores of a porous polymer matrix may be contained in first and second pores of the polymer matrix. First and second pores may be physically and/or spatially distinct to allow differential loading. Optionally, for example, first pores may contain (lyophilized sample comprising) at least a first molecule of interest and second pores may contain (lyophilized sample comprising) at least a second molecule of interest, wherein the second molecule is different from the first molecule. In some embodiments, a lyophilized material may be contained in at least pores of a first porous polymer matrix and pores of a second polymer matrix. Optionally, for example, pores of a first polymer matrix may contain (lyophilized sample comprising) at least a first molecule of interest and pores of a second polymer matrix may contain (lyophilized sample comprising) at least a second molecule of interest, wherein the second molecule is different from the first molecule. In some embodiments, a first polymer matrix and a second polymer matrix (e.g., in the form of a first sheet and second sheet, respectively) may be in contact with one another or may be adjacent to one another, optionally separated by an affinity matrix or other separating material. A polymer matrix (e.g., a first and/or second polymer matrix) may be at least partially covered by, positioned on or enclosed within a layer of impermeable material.

The present disclosure also provides, in some embodiments, a porous polymer matrix containing within its pores a lyophilized sample, the lyophilized sample comprising one or more molecules of interest. For example, a porous polymer matrix may comprise or define pores (e.g., pores internal to the polymer matrix), wherein the pores, according to some embodiments, are partially or completely occupied by or filled with a sample solution. A porous polymer matrix may have any desired shape or form including, for example, the form of a film, a sheet, a strip, a tape, a block, a cylinder, or a bead. First and second pores may be physically and/or spatially distinct to allow differential loading. In some embodiments, first pores may contain (lyophilized sample comprising) at least a first molecule of interest and second pores may contain (lyophilized sample comprising) at least a second molecule of interest, wherein the second molecule is different from the first molecule. In some embodiments, the porous polymer matrix may be combined with a second porous polymer matrix. A lyophilized material may be contained in at least pores of the porous polymer matrix and pores of the second polymer matrix. Optionally, for example, pores of a first polymer matrix may contain (lyophilized sample comprising) at least a first molecule of interest and pores of a second polymer matrix may contain (lyophilized sample comprising) at least a second molecule of interest, wherein the second molecule is different from the first molecule. In some embodiments, the polymer matrix and the second polymer matrix (e.g., in the form of a first sheet and second sheet, respectively) may be in contact with one another or may be adjacent to one another, optionally separated by an affinity matrix or other separating material. A polymer matrix (e.g., a first and/or second polymer matrix) may be at least partially covered by, positioned on or enclosed within a layer of impermeable material.

According to some embodiments, disclosed materials and methods (e.g., lyophilized sample preparation methods, lyophilized samples, and polymer matrices comprising lyophilized samples) may include or may be used with any desired molecule of interest and/or any desired sample. A molecule of interest, for example, may be selected from a protein (e.g., an enzyme, an antibody), a nucleic acid (e.g., a DNA or RNA), a nucleoside triphosphate (or a plurality of nucleoside triphosphates), and an affinity tag or label. Disclosed materials and methods may include or may be used with two or more molecules of interest (e.g., a protein, a nucleic acid, a nucleoside triphosphate, an affinity tag or label, and combinations thereof), according to some embodiments. In some embodiments, a sample may comprise a DNA polymerase, a reverse transcriptase, one or more nucleic acids (e.g., primers or probes), and combinations thereof. For example, a sample may comprise a DNA amplification reagent mastermix or a reverse transcription mastermix (e.g., a reverse transcriptase quantitative PCR (RT-qPCR) master mix). In some embodiments, a sample may further comprise one or more excipients and/or sugars (e.g., non-reducing sugars, reducing sugars) in an aqueous buffer. For example, a sample may further comprise one or more sugars selected from glucose, lactose, sorbitol, mannitol, raffinose, sucrose, trehalose, stachyose, verbascose, isomaltose, dextrin, cyclodextrin, maltodextrin, dextran, branched dextran, and cyclodextran. Non-reducing sugars may function as glassing agents during lyophilization. A sample may comprise one or more non-reducing sugars, for example, non-reducing sugars selected from lactose, sorbitol, mannitol, raffinose, sucrose, trehalose, stachyose, verbascose, isomaltose, cyclodextrin, maltodextrin, dextran, branched dextran, and cyclodextran. Optionally and independently, each sugar (e.g., each non-reducing sugar) may be present (e.g., in a sample solution prior to lyophilization) at a concentration of 0.1-20 wt. %, for example, 0.5-12 wt. %. Optionally, all reducing sugars, all non-reducing sugars, or all sugars (reducing and non-reducing) may be present (e.g., in a sample solution prior to lyophilization) at a total concentration of 0.1-20 wt. % or 0.5-12 wt. %.

In some embodiments, disclosed porous materials and methods (e.g., lyophilized sample preparation methods, lyophilized samples, and polymer matrices comprising lyophilized samples) may include or may be used with pores each having a size (e.g., a longest dimension) of 5-200 microns or may have an average size (e.g., an average longest dimension) of 5-200 microns. In some embodiments disclosed porous materials and methods (e.g., lyophilized sample preparation methods, lyophilized samples, and polymer matrices comprising lyophilized samples) may have a porosity (pore volume) of 25%, 50%, 70%, or 25%-70% (e.g., v/v percent pore volume relative to total matrix volume). In some embodiments, disclosed porous materials and methods (e.g., lyophilized sample preparation methods, lyophilized samples, and polymer matrices comprising lyophilized samples) may comprise or consist of or be used with (a) a sintered synthetic polymer (e.g., high density polyethylene) or (b) a porous polymer selected from polyester, polyethylene, polypropylene, polyurethane, polytetrafluoroethylene, polyvinyl, polyvinylidene fluoride, polyvinyl chloride, polyacrylate, polycarbonate, acrylonitrile butadiene styrene, polyamides (such as nylon), polyolefins, cellulose acetate, polylactic acid, polyphenylene sulphide, toluene diisocyanates, methylene diphenyl diisocyanates, and hexamethylene diisocyanate or (c) a porous polymer selected from fluoropolymers, polyamides, polyethylenes, polypropylenes, polyesters, polyacrylonitriles, polyether imides, polyether ketones, polysulfones, polyethersulfones, polyvinyl chlorides, copolymers of vinyl chloride and acrylonitrile or (d) any combination thereof. For example, a porous polymer matrix may comprise or consist of nylon, polyethylene, or polypropylene or may comprise or consist of a naturally occurring polymer (e.g., an alginate, cellulose, or gelatin polymer). Selection of polymer(s) for use in preparing a composition may be guided by the molecule of interest and/or reaction of interest. For example, polypropylene, polycarbonate, nylon, and, in each case, derivatives thereof along with glass and other borosilicates have broad compatibility with enzymes and other reagents for and reactions involving synthesis and/or manipulation of DNA and RNA (e.g., amplification, sequencing, ligation, restriction, transcription, translation, and methylation (or other modification) reactions.

The present disclosure further provides, in some embodiments, a reaction vessel. A reaction vessel may comprise, for example, any of the lyophilized samples disclosed herein and/or any or the porous polymer matrices disclosed herein. A reaction vessel may comprise one or more molecules of interest. For example, the one or more molecules of interest may be present in the lyophilized sample and/or polymer matrix included in the reaction vessel. A reaction vessel may comprise one or more walls defining a reaction chamber. A reaction vessel may comprise one or more openings in the wall(s), the opening(s) configured, for example, to allow or facilitate entry and/or egress of materials (e.g., fluid materials, solid materials) to or from the reaction chamber. A reaction chamber may be included in a reaction vessel, a reaction container (e.g., a tube, a microtiter plate well) and/or a test device.

The present disclosure further relates to kits. A kit, according to some embodiments, may comprise (a) one or more lyophilized samples (i) comprising one or more molecules of interest and (ii) included in a porous polymer matrix and/or (b) one or more porous polymer matrices each comprising a lyophilized sample each sample having one or more molecules of interest, and/or (c) an aqueous solution for rehydrating the lyophilized sample(s) comprising the one or more molecules of interest, and/or (d) one or more reaction vessels and/or containers. Kits may include (in the aqueous solution or separately) one or more buffering agents, excipients, salts, enzymes, inhibitors, aptamers, adapters, probes, primers, and/or nucleoside triphosphates.

The present disclosure further relates to reconstitution methods and workflows. For example, a method may comprise contacting an aqueous solution and a lyophilized sample included in a porous polymer matrix or contacting an aqueous solution and a porous polymer matrix comprising a lyophilized sample, in each case, to produce a hydrated lyophilized sample (e.g., a hydrated lyophilized sample comprising one or more molecules of interest). A hydrated lyophilized sample may be a rehydrated lyophilized sample solution. In some embodiments, contacting may further comprise contacting the aqueous solution and a reaction chamber, reaction container or reaction vessel comprising the lyophilized sample included in a porous polymer matrix or comprising the porous polymer matrix comprising a lyophilized sample. In some embodiments, a reaction container may be configured to have two or more stages with each stage independently having a lyophilized sample included in a porous polymer matrix or having a porous polymer matrix comprising a lyophilized sample, each independently having one or more molecules of interest. The molecule(s) if interest in each stage may be the same or different. Where the molecules in a first stage differ from the molecules in a second stage and the first stage and the second stage are in fluid communication with one another, a method may comprise contacting the first stage and an aqueous solution to produce a first rehydrated lyophilized sample solution and contacting the second stage and the first rehydrated lyophilized sample solution to produce a second rehydrated lyophilized sample solution. In some embodiments, at least one molecule of interest in a first stage or a second stage may be an enzyme and/or at least one molecule of interest in a second stage or a first stage may be a substrate for the enzyme and/or the second rehydrated lyophilized sample solution may comprise an enzyme and one or more of its substrates. In some embodiments, a method comprises adding an aqueous solution to a reaction container wherein the reaction container comprises at least a first porous polymer matrix and a second porous polymer matrix; optionally wherein the first porous polymer matrix comprises within the pores a lyophilized sample comprising at least a first molecule of interest and the second porous polymer matrix comprises within the pores a lyophilized sample comprising at least a second molecule of interest, different from the first molecule of interest; wherein the method comprises adding aqueous solution to the first and second porous polymer matrices in the reaction vessel, so as to rehydrate the lyophilized samples comprising the first and second molecules of interest. In some embodiments, a method may comprise dispersing a hydrated lyophilized sample (e.g., a rehydrated lyophilized sample solution) comprising one or more molecules of interest.

The present disclosure further relates to assemblies of reaction containers. For example, an assembly may comprise two or more reaction containers, each reaction container independently comprising two or more stages, each stage independently comprising lyophilized sample included in a porous polymer matrix or having a porous polymer matrix comprising a lyophilized sample, with each in turn independently having one or more molecules of interest.

The present disclosure relates to methods for preparing dried materials. For example, a method may comprise dehydrating a sample (e.g., an aqueous sample) in a polymer matrix with a dryer device (e.g., a lyophilization device) to produce a dried sample (e.g., a lyophilized sample) in the polymer matrix, sealing the polymer matrix (e.g., sealing the polymer matrix to limit or prevent rehydration (and limit or prevent further dehydration)). Optionally, sealing may include reversibly bonding the porous polymer to an impermeable membrane (e.g., a liquid impermeable membrane, a fluid impermeable membrane). Sealing, in some embodiments, may comprise sealing the polymer matrix in the dryer (e.g., immediately following drying).

### BRIEF DESCRIPTION OF THE FIGURES

FIG. 1 shows an example embodiment of sheet assembly wherein a sheet of porous polymeric matrix, for example, an expanded polymer (e.g., an expanded polyethylene such as Vyon® M, Vyon® T, Vyon® PT, Vyon® D, Vyon® F, Vyon® HP) may be layered on a support (e.g., a support membrane or composite, for example, a polypropylene or foil coating) prior to placement on a shelf in a drying unit for lyophilization. A sample solution may be included in a porous polymer matrix before or after association with (e.g., layering on) a support.

FIGS. 2A-2D show example embodiments of how a sample may be added to a porous polymeric matrix. FIG. 2A illustrates pipette application on the surface for penetration into the pores. FIG. 2B illustrates nano-dispenser application (e.g., ink-jet printing) on the surface for penetration into the pores. FIG. 2C illustrates soaking in a dip tank. FIG. 2D illustrates roller application. Each porous polymeric matrix of FIGS. 2A-2D optionally may include a support.

FIG. 3 shows an example embodiment of drying unit 310 comprising multiple shelves. A drying unit (shelf stack) may comprise thermal conductive materials (e.g., metal, carbon filled plastic, CoolPolymer®, or similar material having a low thermal mass for fast drying) or otherwise may be thermally conductive. Drying unit 340 loaded with assembly 330 may be placed in freeze-drying oven 360, for example, where the drying unit is in thermal union with a shelf of the freeze-drying oven. Assemblies 330 include a porous polymer matrix and a support.

FIG. 4 shows an example embodiment of how the drying unit (shelf stack) may be enclosed by enclosure 450 and sealed prior to insertion into the oven. Enclosure 450 includes walls defining an opening configured to admit drying unit 440. Seal interface 451 engages enclosure 450 and a shelf of freeze-drying oven 460.

FIG. 5 shows an example embodiment of how sheet assemblies 530 containing lyophilized molecules of interest may be removed from oven 560, enclosure 550, and drying unit 540 and formed (e.g., stamped or cut using press 570) into desired sizes and shapes 530a, 530b, 530c for insertion into example test device 580. Test device 580 may include an opening for accepting a fluid (e.g., a bodily fluid, a test fluid). An accepted fluid may contact a portion of an assembly in device 580. Test device 580 may include one or more viewing windows, for example, where the contact is associated with a visible display (e.g., movement of the fluid itself) and/or a conditional visible display (e.g., a visible product of a reaction between the molecule of interest and a component of the sample if present).

FIG. 6 shows an example embodiment of how a tape can be made that can be rolled into a cylinder before drying and enclosed in a suitable cylindrical sealed container.

FIG. 7 shows an example embodiment of a cylindrical container for sealing a tape spool for lyophilization.

FIG. 8A shows a micrograph of an example porous polymer matrix. FIG. 8B shows a micrograph of an example porous fiber polymer matrix. FIG. 8C shows a micrograph of an example polyurethane flexible porous foam polymer matrix. FIG. 8D shows a micrograph of an example polytetrafluoroethylene flexible porous hydrophobic polymer matrix.

FIGS. 9A-9F illustrate example embodiments of porous polymer matricies. FIG. 9A shows a Vyon® PPF polymer matrix of 4.75 mm thickness before lyophilization. FIG. 9B shows a Vyon® PPF polymer matrix of 4.75 mm thickness after lyophilization. FIG. 9C shows a close-up (micrograph) of the material of FIG. 2B. FIG. 2D shows a Vyon® PPF polymer matrix of 2 mm thickness before lyophilization. FIG. 9E shows a Vyon® PPF polymer matrix of 2 mm thickness after lyophilization. FIG. 9F shows a close-up (micrograph) of the material of FIG. 2E.

FIGS. 10A-10D illustrate embodiments of porous polymer matrices. FIG. 10A illustrates a porous polymer matrix starting material with empty pores. This starting material may be contacted with a solution comprising molecules of interest (e.g., one or more enzymes) and, optionally, excipients that penetrates to fill or at least partially fill the pores with the solution. This loaded matrix may be frozen with solution in pores. The frozen material may dry, for examply, by sublimation. Water vapor may escape via pore channels of the matrix. Freeze-dried excipients may for a stable, dry sub-matrix within pores of the polymer matrix and contain the molecules of interest. The resultant polymer matrix mat may be formed (e.g., cut, rolled, punched or otherwise shaped) into one or more desired shapes and/or proportions to contain a desired quantity of the molecule(s) of interest.

FIG. 1013 illustrates an example embodiment in which a lyophilized material in a porous polymer (e.g., produced according to FIG. 10A and/or as otherwise disclosed herein) may be rehydrated and dispensed in a tube (e.g., a tube separate from the tube in which the material was freeze dried). The rehydrated solution may carry out a reaction in the separate tube.

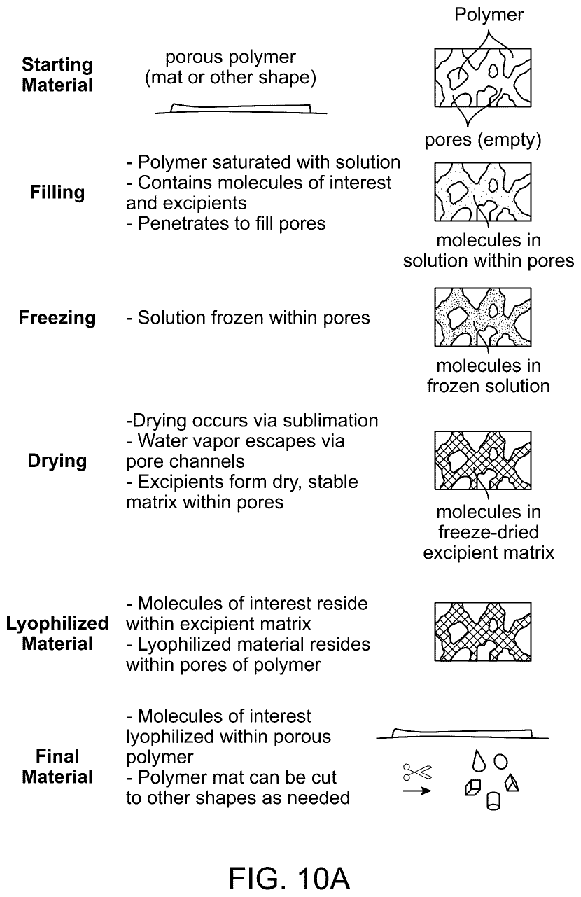

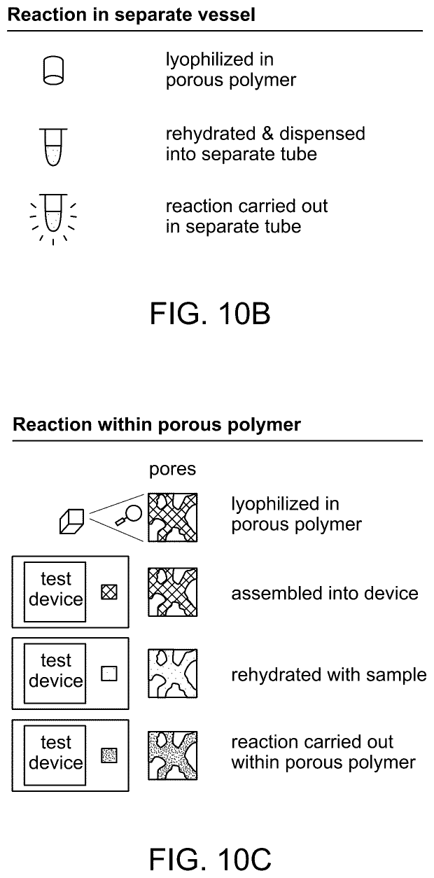

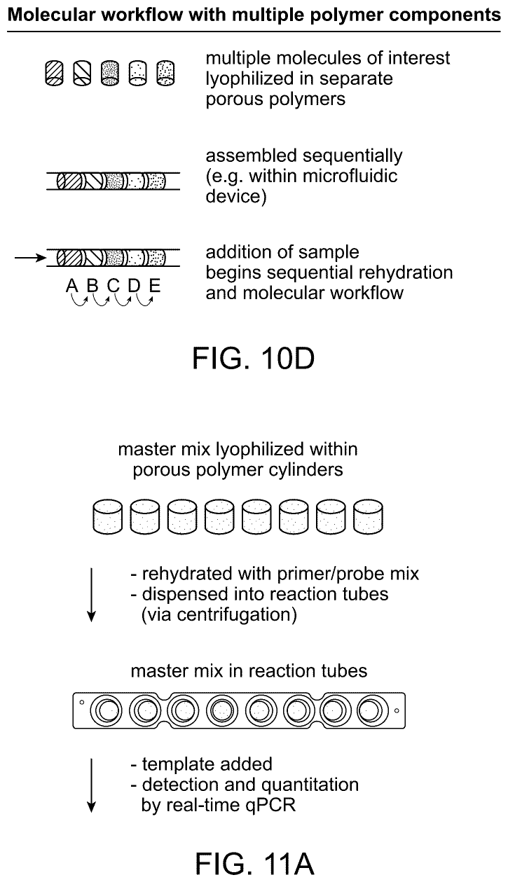

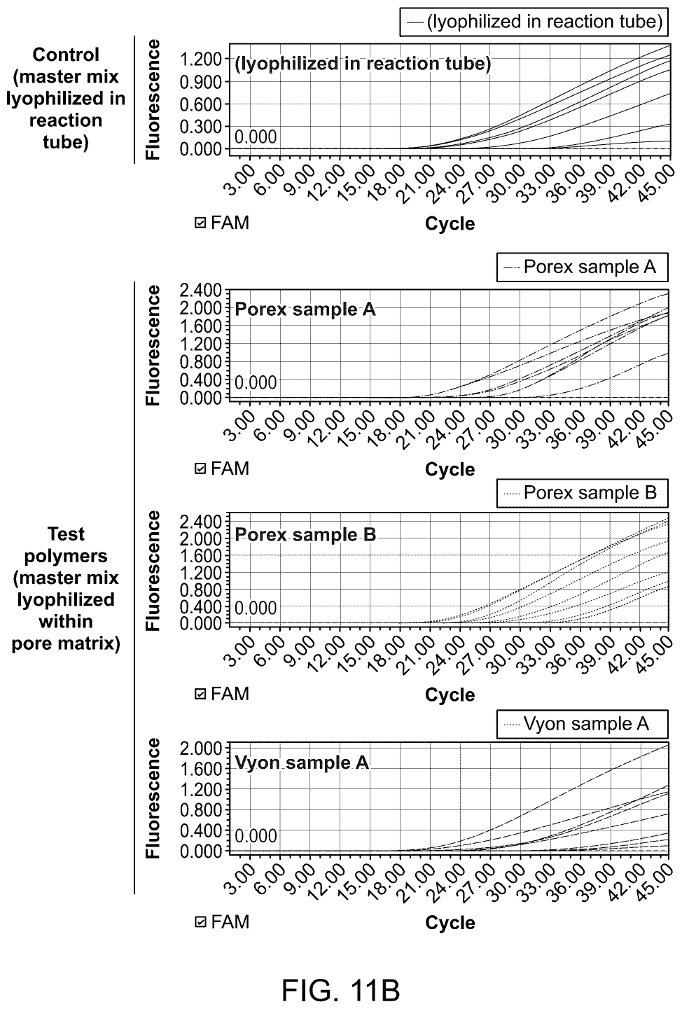

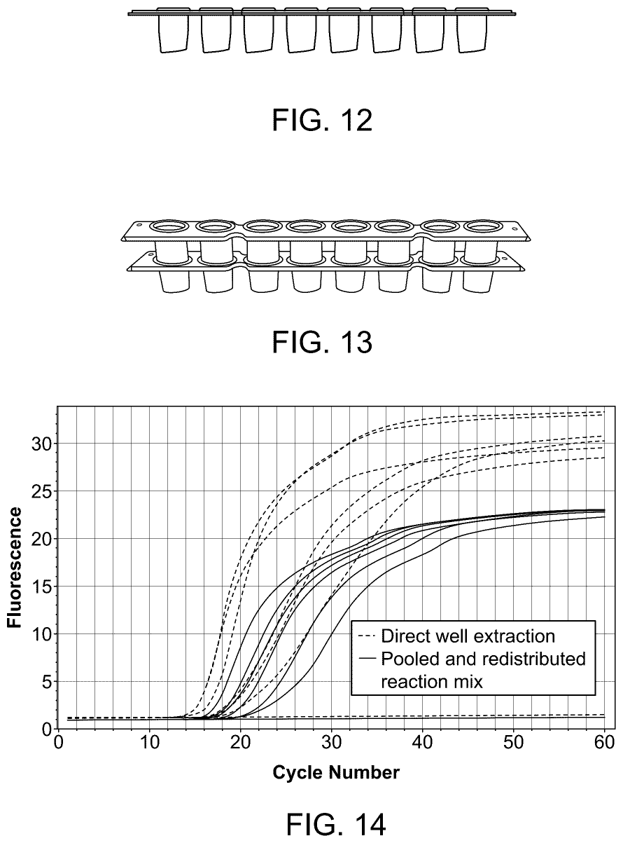

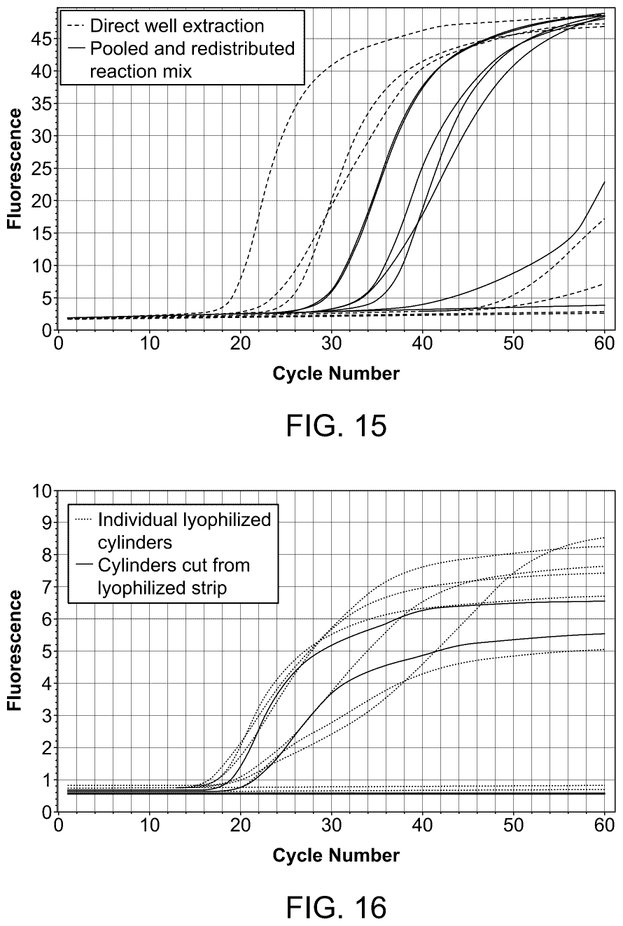

FIG. 10C illustrates an example embodiment in which a reaction (e.g., a desired reaction) occurs in the polymer matrix pores on addition of substrate to lyophilized sample.

FIG. 10D illustrates an example embodiment of a workflow that makes use of five separate lyophilized polymer matrices, each with one or more molecules of interest. The separate matrices may be assembled in a desired sequence (e.g., in a microfluidic device). A fluid may be contacted with a first lyophilized matrix to rehydrate its molecule(s) of interest. The molecules or reaction products of these molecules may be wicked or otherwise conveyed to the second matrix to contact the molecule(s) if interest in the second matrix. This may continue in sequence until all five matrices are rehydrated and the desired product is produced. In some embodiments, a cylinder comprising multiple polymer layers that each contain the molecules of interest required for a reaction with substrate may be combined in a workflow. Multiple polymer layers may be punched out of multiple stacked polymer sheets to form a cylinder for insertion into a microfluidic device. Multiple stacked sheets as described herein might be utilized in a test strip or fabric without utilizing a microfluidic device. In some embodiments, an assembly of multiple polymer layers may be combined with one or more similar (or different) assemblies to form an array for parallel manufacturing or analytical purposes.

FIGS. 11A-11B show functional recovery after lyophilization in a polyethylene matrix, using a Cirrus' DNA Master Mix (Fluorogenics Ltd., UK, legacy product #CIR-DNA-1000-01) dosed into test cylinders of porous polyethylene materials and then lyophilized. Cirrus DNA Master Mix is a lyophilized qPCR master mix (vial format), and thus contains all excipient components required for stable freeze-drying.

FIG. 11A shows an example embodiment of lyophilized polymer matrices. A master mix solution was lyophilized in a porous polymer matrix. Lyophilized master mix material was formed into cylinders. Master mix was rehydrated with primer/probe mix and dispensed into reaction chambers (via centrifugation). Template was may be added and detection and quantification of amplification products was performed by real-time qPCR.

FIG. 11B shows real-time qPCR data with fluorescent detection of amplification demonstrating functional recovery from an example amplification reaction performed according to FIG. 11A. The uppermost panel shows results of a control in which the master mix was simply lyophilized in the ultimate reaction tube. The remaining panels show results of assays with master mix lyophilized in a Porex® A, Porex® D or hydrophilic Vyon® F porous matrix.

FIG. 12 shows an example extraction strip that may be used to contain solid phase amplification (SPA) cylinders.

FIG. 13 shows an example extraction strip and test strip used to extract master mix from resuspended SPA cylinders.

FIG. 14 shows example SPA Material A RD8074 amplification curves, dye added as part of reagent dissolution. (solid lines=pooled and redistributed reaction mix, dashed lines=Direct well extraction).

FIG. 15 shows example SPA Material B RD8074 amplification curves, dye added as part of reagent dissolution (solid lines=pooled and redistributed reaction mix, dashed lines=Direct well extraction).

FIG. 16 shows example SPA Material A RD8075 amplification curves, dried with DNA binding dye within the reagent. (dashed lines=individual lyophilized cylinders, solid lines=cylinders cut from lyophilized strip).

### DETAILED DESCRIPTION

### General Considerations

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a”, “an”, and “the” include plural referents unless the context clearly dictates otherwise. For example, the term “a protein” refers to one or more proteins, i.e., a single protein and multiple proteins. The claims can be drafted to exclude any optional element when exclusive terminology is used such as “solely,” “only” are used in connection with the recitation of claim elements or when a negative limitation is specified.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which may be readily separated from or combined with the features of any of the other several embodiments without departing from the scope or spirit of the present teachings. Any recited method can be carried out in the order of events recited or in any other order which is logically possible.

Numeric ranges are inclusive of the numbers defining the range. All numbers should be understood to encompass the midpoint of the integer above and below the integer i.e. the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “container” refers to a human-made container. A container may comprise one or more walls (e.g., defining an interior volume) and optionally one or more openings. Containers comprising one or more openings may further comprise one or more closures (e.g., a removable closures) for some or all such openings. A closure optionally may comprise an aperture or a septum, for example, to provide fluid communication with a volume of the container and a connected or inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze-dried, crystalline, aqueous, and (e) having one or more additional components beyond those found in nature (e.g., buffering agents, a detergent, a dye, a solvent or a preservative).

All publications, patents, and patent applications mentioned in this specification are herein incorporated by reference to the same extent as if each individual publication, patent, or patent application was specifically and individually indicated to be incorporated by reference.

Methods are described for preparing a lyophilized sample comprising one or more molecules of interest. Such methods comprise (i) adding a sample comprising one or more molecules of interest in solution to a porous polymer matrix and allowing the sample to penetrate into the pores of the porous polymer matrix; and (ii) lyophilizing the sample within the pores of the porous polymer matrix thereby preparing a porous polymer matrix containing the lyophilized sample.

### Lyophilized Samples

The methods can be used to prepare lyophilized samples containing any one or more molecules of interest. Present embodiments include samples comprising biological molecules such as proteins, nucleic acids, or nucleoside triphosphates. For example, in one embodiment, one or more molecules of interest are selected from the group consisting of a protein, a nucleic acid, and one or more nucleoside triphosphates. The sample may comprise two, three, four, five or more molecules of interest, which may include one or more proteins, nucleic acids, or nucleoside triphosphates.

Examples of proteins of interest include enzymes, such as RNases, DNases, deaminases, dioxygenases, glucosyl transferases, ligases, polymerases, reverse transcriptases, exonucleases, kinases, restriction endonucleases, nicking endonucleases, methylases, glycosylases, glycosylase lyases, endonucleases, glycosidases, terminal transferases, transposes, primases, telomerases. In some embodiments, the one or more molecules of interest is or includes a DNA polymerase or reverse transcriptase. Examples of nucleic acids of interest include one or more oligonucleotide reagents such as primers or probes. Nucleoside triphosphates of interest may include any naturally occurring or synthetic nucleoside triphosphate. Examples of naturally occurring nucleoside triphosphates are rNTPs and dNTPs, including dUTP, dTTP, dGTP, dCTP, dATP rUTP, rTTP, rGTP, rCTP, and rATP.

In some embodiments, a sample to be lyophilized comprises all or a subset of reagents involved in a reaction of interest, and therefore the method is for preparing a lyophilized reagent or reagent mastermix comprising all or a subset of reagents involved in a reaction. In some embodiments, a sample for lyophilization may include a subset or all reagents for DNA library preparation such as ligases, adaptors, etc. In other embodiments, samples for lyophilization may include a subset or all reagents for synthetic biology nucleic acid assembly such as restriction endonucleases etc.

For example, the sample may include a subset of (i.e., one or more) or all reagents (master mixes) involved in DNA amplification such as loop-mediated isothermal amplification (LAMP), polymerase chain reaction (PCR), reverse transcription, transcription-mediated amplification (TMA), nucleic acid sequence-based amplification (NASBA), ligase chain reaction (LCR), strand displacement amplification (SDA). Individual reagents for amplification may include for example, Hot Start Taq DNA Polymerase, Bst polymerase and variants thereof, WarmStart® DNA Polymerase, WarmStart RTx Reverse Transcriptase and WarmStart Luna® Reverse Transcriptase (New England Biolabs, Ipswich, MA).

The sample that is absorbed by a porous polymer matrix prior to lyophilization may contain in addition to molecules of interest, one or more buffers as defined herein. The buffer may be an aqueous buffer, such as used in standard commercial enzyme reactions (see New England Biolabs, Ipswich, MA). Alternatively, the sample may include an organic solvent instead of water such as an oil.

### Excipients

A sample may further comprise one or more excipients. Excipient(s) may aid lyophilization within the porous polymer matrix. Suitable excipients for use in the embodiments disclosed herein include those generally known in the art for aiding lyophilization. For example, the sample may comprise one or more sugars, such as glucose, lactose, sorbitol, mannitol, raffinose, sucrose, trehalose, stachyose, verbascose, isomaltose, dextrin, branched dextran, cyclodextrin or maltodextrin; optionally at a concentration of 0.1-20 wt. %, such as 0.5-12 wt %.

Excipients may include carbohydrates such as dextrans, dextrin, branched dextran (polyglucose) e.g. Ficoll and cyclodextrin(s) or 0.1-10%, starch, etc. Other excipients may include any one or more of Polyenols, such as PEG 0.1-10 wt. %, Poly vinylpolypyrrolidine (PVP) 0.1-10 wt. %, Anti Maillard reagents such as Threonine 1-20 mM, and other amino acids, Gelatin 0.1-10 wt. %, BSA-2 to 100 mg/mL. Other excipients may include one or more surfactants e.g. polysorbate(s), dispersants, and/or antifoaming agents. These excipients are preferably used in an amount of from 0.1-10 wt. %, as indicated in some cases above, or at the concentrations indicated above. However, other amounts may also be used.

### Type of Porous Polymer Matrix

Any porous polymer matrix may be used in compositions and methods of the disclosure. Selection of a particular porous polymer matrix may take into consideration whether the polymer matrix contains pores into which the sample solution comprising the one or more molecules of interest can penetrate, the polymer is compatible with the molecule of interest (e.g. does not significantly affect the properties of the molecule), the polymer is compatible with any reaction chemistry of interest (e.g., upon or following rehydration of the molecule of interest), the polymer matrix retains a suitable structure when subjected to lyophilization (freeze-drying) conditions, and/or whether the polymer matrix (e.g., with or without a support) is amenable to manipulation including forming into pieces (e.g. by cutting) once freeze-dried. Factors that may influence compatibility of a polymer and a molecule of interest or a reaction of interest include the purity of the polymer (e.g., the presence of heavy metals), the presence or absence of additives that may be released, the potential for desired or undesired interactions between the molecule of interest, reactants, products, or other components with the matrix (e.g., charge absorption and/or reagent abstraction).

Examples of various porous polymer matrices include sintered polymer matrices, woven polymer matrices, and foam polymer matrices. Porous polymers are available commercially for example from Porvair Sciences (Wrexham, UK) and Porex (Fairburn, GA). They can be made in the form of sintered porous polymer, porous fiber and porous foam.

In considering the reagents for drying, their solvents and their uses, various parameters may be considered such as hydrophobicity, pore size, and porosity. Porosity refers to the percentage of void space in the polymer that is a property of the polymer matrix.

In some embodiments, the porous polymer matrix suitable for lyophilization may have pores having a size in the range of 5-200 microns; for example more particularly in the range of 5-50 microns, 5-100 microns, or 15-150 microns. The pores in the porous polymer matrix may be of substantially the same size or may be of different sizes within the size range, in which case the pore size may be an average pore size. For example, the porous polymer matrix may comprise one layer (e.g. an upper layer) comprising pores of a first size and another layer (e.g. a lower layer) comprising pores of a second size.

The porous polymer matrix may have a porosity of greater than 20% for example, greater than 30% or 40%, 50%, 60%, 70% or 75%. For example, the porosity may be in the range of 25-70%. Pore size and/or porosity may be determined by any desired method including, for example, capillary flow porometry, mercury intrusion porosimetry, x-ray refraction, nanoparticle intrusion, and gas adsorption.

In the example of nucleic acid amplification, both hydrophobic and hydrophilic porous polymers with a pore size in the range of 15 μm-150 μm were demonstrated to be effective for lyophilizing amplification reagents such that they could be stored for at least 45 days, for example at least 75 days, for example at least 100 days at 37° C.

### Porous Polymer Matrix Composition

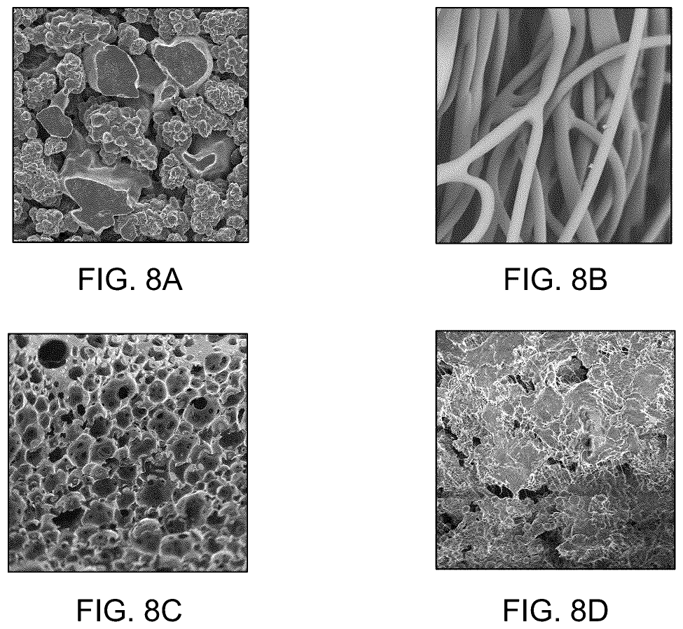

The porous polymer matrix may be, for example, formed by sintering, from fibers or in a solid foam (see for example FIG. 8).

The polymer matrix may comprise or consist of a synthetic polymer. Examples of synthetic polymers useful in the disclosures herein include polyester, polyethylene, polypropylene, polyurethane, polytetrafluoroethylene, polyvinyl, polyvinylidene fluoride, polyvinyl chloride, polyacrylate, polycarbonate, acrylonitrile butadiene styrene, polyamides (such as nylon), polyolefins, cellulose acetate, polylactic acid, polyphenylene sulphide, toluene diisocyanates, methylene diphenyl diisocyanates, and hexamethylene diisocyanate; or fluoropolymers, polyamides, polyethylenes, polypropylenes, polyesters, polyacrylonitriles, polyether imides, polyether ketones, polysulfones, polyethersulfones, polyvinyl chlorides, and copolymers of vinyl chloride and acrylonitrile; or any combination thereof. In one embodiment, the porous polymer matrix comprises or consists of polyethylene, polypropylene, or nylon.

Alternatively or additionally, the polymer matrix comprises or consists of a naturally occurring polymer, such as an alginate, cellulose, or gelatin polymer.

In some embodiments, the polymer matrix comprises a single polymer (e.g., polyethylene, polypropylene, polytetrafluoroethylene) or a composite. A polymer matrix may be hydrophobic or hydrophilic. Example polymers include Vyon® porous polymers (Porvair Sciences, Wrexham, UK)) and Porex® porous polymers (Porex, Fairburn, GA) described in the examples. Vyon® polymers include breathable polymers of sintered high density polyethylene with a generally uniform distribution of pores with a semirigid structure such that it holds up to manipulation and cutting. Vyon® polymers shed few fibers and are generally stable across a broad temperature range (e.g., −70° C.-80° C.). Additional information on the composition and properties of specific Vyon® polymers (e.g., Vyon® M, Vyon® T, Vyon® PT, Vyon® D, Vyon® F, Vyon® HP, Vyon® PPD, Vyon® PPF, Vyon® PP-HP, and Vyon® PTFE) may be obtained from the manufacturer. Porex® A, Porex® B, and Porex® D are porous hydrophilic polyethylene matrices and Porex® C, is a is a porous hydrophilic polypropylene matrix, all of which may be easily manipulated including winding into a rolled form. Average pore sizes are 15-50 μm for Porex® A, 15 μm for Porex® B, and 90-170 μm for Porex® D. Additional information on porous hydrophilic and hydrophobic Porex® matrices may be obtained from the manufacturer.

The porous polymer matrix for receiving the sample in its pores for lyophilization may consist of a single type of polymer. Alternatively, the porous polymer matrix may contain two or more different types of polymers that are combined in a single porous polymer matrix or geographically separated, optionally in a multi-layer format (e.g. a first layer of a first polymer and a second layer of a second polymer; e.g. an upper layer of a first polymer and a lower layer of a second polymer).

Moreover, where the reagents are light sensitive, it may be desirable to include a light filter into the polymer matrix. It may also be desirable to modify the thermal stability of the polymer matrix so obtain the optimum temperature for drying reagent from either oil based or aqueous solutions or suspensions of reagents.

In various embodiments, the polymer matrix may contain a color suitable for ease of use. For example, a first reagent containing a mixture of enzymes for amplification, such as a polymerase and a reverse transcriptase, may be lyophilized within a first polymer matrix (or first region of the polymer matrix) that has a first color (e.g. blue) and a second reagent including primers and probes may be included in a second polymer matrix that has a different color (e.g. yellow). In this way the two different colored polymer matrices, containing different reagents, may be easily distinguished from each other—e.g., when stored together.

In some embodiments, the porous polymer matrix to which the sample is added is in the form of a block (e.g. cube), a cylinder, or a sphere (e.g. bead). Alternatively, the porous polymer matrix to which the sample is added may in the form of a sheet (of any shape), strip, or tape, which may be substantially flat. In the context of a porous polymer matrix, the term flat or substantially flat refers to a porous polymer matrix that has a smooth, level surface without raised areas or indentations.

### Adding Sample to the Polymer

The sample comprising one or more molecules of interest may be added to the porous polymer matrix by any means so long as the sample penetrates into the pores of the porous polymer matrix. For example, the sample solution may be poured, sprayed, or spread over the surface of the matrix. Absorbance volume of the sample into the polymer matrix refers to the amount of sample that can be taken into the polymer. Absorbance volume may be measured to determine the dispersion and dose of sample in the polymer prior to lyophilization. Pores of a polymer matrix may be at least partially filled by a sample solution. The degree to which pores are filled may vary on the bases of pore size, pore distribution, pore shape, composition of the polymer matrix (e.g., presence, valence, charge and density of charged functional groups; presence and kind of hydrophobic and/or hydrophilic functional groups), and sample solution composition (e.g., presence, valence, charge and concentration of charged species; presence and kind of hydrophobic and/or hydrophilic solvents and/or functional groups). In some embodiments, a sample solution and a polymer matrix may be paired with one another to achieve a desired level of sample solution retention. Pairing sample solution and polymer matrix may include selecting or formulating a sample solution and/or a polymer matrix to achieve the desired retention. Desired retention may be expressed as volume of sample solution that remains associated with a polymer matrix after contact (e.g., dipping, soaking, vacuum-infiltration) and excess sample solution has been removed (e.g., by draining, pipetting, siphoning, or flowing excess solution away from a matrix). This may be assessed, for example, by subtracting the volume of sample solution removed from the volume of the sample solution initially contacted with the matrix.

In some embodiments, it may be desirable to enhance dispersal of the sample within the polymer matrix, for example so as to achieve an even distribution of the one or more molecules of interest throughout the porous polymer matrix. This may be achieved by adding a dispersant or effervescent agent to the sample prior to lyophilization. Alternatively the polymer matrix may comprise a polymer having dispersant properties (e.g., anionic or cationic polymers including polyacrylic acid, fatty acids and derivatives, phosphate esters, acetylene diols, soya lecithin, polyolefin, maleic acid-modified polypropylene).

A layer of impermeable material may be positioned underneath the porous polymer matrix to support the porous matrix and prevent the sample solution from permeating out of the porous polymer matrix, e.g. prior to lyophilization.

In some embodiments, a defined volume of sample having a known concentration of the one or more molecules of interest is added into the porous polymer matrix. Lyophilization preferably results in an even distribution of sample throughout the polymer. The amount or concentration of the molecule(s) of interest in the sample, or the size or volume of the porous polymer matrix into which the sample is to be added, may be adjusted in order to achieve the desired concentration of the molecule(s) of interest within the porous polymer matrix. The volume of the polymer matrix, as determined by size and thickness, can be readily customized to the dose of reagent required in each reaction for which the reagent is required. Customization can also be achieved with respect to robot handlers and receiving reaction compartments used for reactions or workflows such as microfluidics. In some embodiments, the mass or volume of a lyophilized porous polymer matrix to be used for a given application may be determined empirically and/or defined by a predetermined formula.

### Lyophilization Process

A composition (e.g., a sample solution—porous polymer matrix composition) may be lyophilized by any desired method. For example, following addition and penetration of sample into pores of a porous polymer matrix, the sample is lyophilized within the pores of the porous polymer matrix thereby preparing a porous polymer matrix containing the lyophilized sample.

Lyophilization is the process of drying a mixture utilizing sublimation. This is carried out by reducing the pressure such that the water in the frozen formulation is removed without the formation of a liquid phase. Considerations in selecting lyophilization conditions include the eutectic point and collapse temperature of the composition to be lyophilized (e.g., a sample solution, a porous polymer matrix, and combinations thereof). In this context, the eutectic point is the lowest temperature at which the liquid phase of composition at a given pressure is stable. A eutectic composition may be a solid having a superlattice and may melt or solidify at a temperature that is lower than any of the components of the composition alone. In this context, the collapse temperature is the is the temperature at which the material will collapse under vacuum. These two parameters affect the pre-vacuum and primary drying freeze temperature variables respectively. Considerations may also include, in the context of lyophilization time and/or pressure selection at other drying stages (e.g., secondary stages), the physical geometry of the material, such as total volume and volume to surface area ratio of the composition-polymer assembly to be lyophilized. A larger volume and/or a higher volume to surface area ratio may increase the time required for sublimation and therefore the total drying time.

The sample containing molecules of interest are added to the porous polymer matrix and penetrate into the pores between particles of polymer in the matrix. The porous polymer matrix containing the penetrated sample solution within its pores is placed in a freeze-drying apparatus and freeze-drying conditions are applied. The freeze drying process replaces the solvent vapor with air within the pores. When rehydration occurs, the air is once again replaced by solvent. The polymer structure and composition preferably remains unchanged or substantially unchanged throughout the process of freezing, heating dehydration and rehydration. In some embodiments, the manufacturing method could be a continuous or semi-continuous process. Thus, the steps of adding sample, lyophilization, and optionally forming into pieces (discussed below), may be performed in a single continuous or semi-continuous process.

### Storage Following Lyophilization

Following lyophilization, the pores of the polymer matrix contain the lyophilized sample comprising the one or more molecules of interest. The polymer matrix comprising the lyophilized sample can then be conveniently stored until the molecule of interest is required for use. Thus, the polymer matrix may be considered to be a convenient storage medium for the lyophilized sample, which is contained within the pores of the polymer matrix. For example, if the porous polymer matrix is in the form of one or more flat sheets, or strips, it may be stored flat, for example as multiple layered flat sheets or strips. Alternatively, if the porous polymer matrix is in the form of a tape, it may be stored as a roll (e.g. rolled into a cylinder shape), or wound around a spindle or into a cassette.

Advantages of lyophilization of sample within a porous polymer matrix that is a sheet, strip or tape, which typically are substantially flat, include one or more of the following:
(a) Increase in capacity of a drying oven by stacking of two or more sheets or strips in a freeze-drying apparatus for simultaneous lyophilization.
(b) Once dried two or more substantially flat polymer matrices comprising the same or different lyophilized samples can be stacked on top of each other for ease and efficiency of storage.
(c) Providing customized doses by punching or cutting pieces of the strip or sheet prior to use.
(d) If the substantially flat porous polymer matrix is in the form of a flexible tape or strip, following addition of the sample, the tape or strip can be passed/wound through a freeze-drying apparatus for lyophilization in a discontinuous or continuous process; and/or once dried the polymer matrix tape or strip comprising the lyophilized sample can be stored in a roll, for example spooled into a cassette, for ease and efficiency of storage.

In some embodiments, following lyophilization, the polymer matrix containing the lyophilized sample (or the one or more pieces of polymer matrix) may be reversibly covered by or encased within a layer of impermeable material, to prevent rehydration until desired. Encasement may be achieved by a membrane or film associated with an adhesive or plastic sheeting. Polymer matrix comprising lyophilized sample (e.g. in the form of a strips or rolled tape) may be placed into hydration resistant bags such as a Mylar® pouch to reduce oxygen and moisture ingress on storage. At the time of use, the polymer matrix (e.g. strip or tape) can be separated or removed from the impermeable material, optionally formed (e.g. cut or punched) into the desired size if this has not already been done, and then hydrated, such as in a suitable reaction tube.

Forming into Pieces

In some embodiments, it may be desirable to form the porous polymer matrix comprising the lyophilized sample into one or more pieces having a desired size, volume, and/or shape. For example, it may be desirable to have a piece of polymer matrix comprising the lyophilized sample of a size, volume, and/or shape that fits into a reaction container (e.g. reaction tube) of corresponding dimensions.

In some embodiments, following lyophilization, the porous polymer matrix can be cut, punched, or fragmented, or otherwise shaped into multiple individual pieces of defined size, volume, and/or shape, each piece containing substantially the same amount of sample/molecule of interest. For example, if the porous polymer matrix is in the form of a sheet, strip or tape, 2-dimensional pieces having shapes such as polygons such as diamonds (for microfluidic applications), circles, or squares etc., can be cut or punched from the sheet, strip or tape resulting in fragments of defined shape and size containing each containing the substantially same amount of sample. Alternatively, the porous polymer matrix may be divided/formed into 3-dimensional pieces such as cubes, cylinders or spheres of defined volume or size each containing the substantially same amount of sample. In the context of this disclosure, shapes are referenced as 2-dimensional. It will be understood that polymer matrix shapes may be thin (e.g., on the order of mm, μm, or nm) and, in that respect, resemble 2-dimensional object, but will generally have a measurable thickness.

The size, volume, and/or shape of the polymer matrix comprising the lyophilized sample may be determined by the dimensions of a reaction container or test device. For example, the shape, volume, and/or size of the one or more reaction containers or test devices may be complementary to the shape, volume, and/or size of the polymer matrix (or piece(s) of polymer matrix), for example, so that the polymer matrix or pieces thereof fit snugly into the reaction container or device, or a compartment thereof.

For example, the pieces may be cut/punched directly into the intended container. If the concentration of the molecule(s) of interest within the polymer matrix is known, it may be desirable to have a piece of polymer matrix comprising the lyophilized sample of defined volume in order to provide a defined amount of the molecule of interest (e.g. an amount of a molecule, such as an enzyme, that is effective for a reaction). The polymer matrix comprising the lyophilized sample may be formed into one or more pieces having the desired size, volume, and/or shape by any means. For example, the one or more pieces may be cut, stamped, punched torn or fragmented (e.g., along one or more pre-scored or perforated lines or zones) from the polymer matrix; or the polymer matrix may be fragmented into multiple pieces. The one or more pieces of polymer matrix comprising the lyophilized sample may be, for example, in the form of one or more strips, circles, squares, cubes, cylinders, spheres (e.g. beads), or any other shape, as desired by the user or determined by the dimensions of a reaction container.

For example, the reagent is a master mix that has been air dried or freeze-dried (lyophilized) within a 3-dimensional porous polymer matrix (e.g. in the form of a cylinder) and then inserted into a reaction vessel, so that the addition of buffer to the porous polymer matrix in the reaction vessel, optionally followed by a centrifugation step, releases the master mix into the reaction compartment.

In one example, the reagent is a PCR master mix, that has been air dried or freeze-dried (lyophilized) within a porous polymer matrix cylinder that is inserted into a PCR reaction compartment that is a tube or well, so that the addition of buffer to the cylinder in the reaction compartment and a centrifugation step releases the dose into the reaction compartment.

### Use of Multiple Polymer Matrices

In some embodiments, multiple polymer matrices each containing a different lyophilized sample within the pores (optionally identifiable by different colors) may be presented in a stacked e.g. multi-layer format from which pieces containing dosage volumes of each reagent/molecule of interest may be cut, stamped, punched torn or fragmented (e.g., along one or more pre-scored or perforated lines or zones) into one or more reaction containers or test devices.

An example of the use of two porous polymer matrices containing different lyophilized reagents for a single reaction might be where one porous polymer matrix contained a polymerase and a second porous polymer matrix contained oligonucleotide primers. In this scenario, the matrices would be cut or punched to an appropriate size and shape and placed in a test device. A substrate in solution added to the test device would rehydrate the lyophilized reagents to permit the reaction to occur.

The lyophilized sample may be contained within the pores of at least a first polymer matrix and a second polymer matrix, different from the first. Optionally, a first polymer matrix contains a first molecule of interest within its pores, and the second polymer matrix contains a different second molecule of interest within its pores. The first and second polymer matrix may be composed of the same or different polymers. Thus, in some embodiments, different molecules of interest are lyophilized within different polymer matrices that can be stored together or separately.

### Rehydration of the Sample

Following lyophilization and optional storage and/or formation into pieces of desired shape and/or size (e.g. volume), methods of the disclosure may further comprise the step of rehydrating the lyophilized sample comprising one or more molecules of interest, for example so that the molecule(s) can be used in a reaction. Rehydration may be achieved by adding an aqueous solution to the polymer matrix that contains the lyophilized sample.

In some embodiments, it may be desirable to release or disperse the molecule(s) of interest from the polymer matrix. Dispersal of the one or more molecules of interest from the polymer matrix upon rehydration may be enhanced, for example, by including a surfactant, dispersant or effervescent agent in the sample prior to lyophilization, or in the aqueous rehydration solution. Alternatively, or in addition, the polymer matrix may comprise a polymer having dispersant properties, e.g. when contacted with aqueous solution,

For example, the polymer matrix may be placed into a reaction vessel and the method comprises adding aqueous solution to the reaction vessel. Rehydration and release of the molecule(s) of interest then proceeds in the reaction vessel.

Following rehydration and release of molecule(s) of interest from the pores of the polymer matrix into the reaction vessel, a further reaction using the molecule(s) of interest (e.g. a DNA amplification or reverse transcription reaction) can then proceed in the reaction vessel. In these embodiments, the rehydration solution may contain one or more additional reagents required for the reaction and/or a substrate for the reaction. For example, the rehydration solution may contain one or more enzymes, buffer components, primers, NTPs, and/or template molecules.

In one embodiment, the reagent assembly could form a part of a consumable that allows the reagent to be transferred to a standard reaction tube. In one example, the reagent is a PCR master mix that has been air dried or freeze dried (lyophilized) within a porous matrix cylinder that is inserted into a PCR reaction compartment that is a tube or well, so that the addition of buffer to the cylinder in the reaction compartment and a centrifugation step releases the dose into the reaction compartment.

Alternatively, an aliquot of the rehydrated sample may be removed from the rehydration reaction vessel into a separate reaction vessel for the subsequent reaction.

In some embodiments of the method, the porous polymer matrix serves not only as a storage means for a molecule or mixture of molecules of interest but also as a reaction vessel. Upon rehydration of the lyophilized sample, the molecule(s) of interest are not released from the polymer matrix but instead remain within the pores or diffuse within the polymer matrix. Alternatively, the rehydrated molecule(s) of interest may diffuse from a first porous polymer matrix to a second porous polymer matrix placed adjacent to the first, e.g. within a reaction vessel or test device.

For example, for an RT-PCR or RT-LAMP reaction, the first polymer matrix contains within its pores, the air dried or freeze-dried (lyophilized) RT-PCR or RT-LAMP master mix. A rehydration solution comprising a target nucleic acid is added directly to the polymer matrix and enters the pores, thereby rehydrating the master mix and allowing the PCR or LAMP reaction to occur within the flexible porous polymer matrix. A color dye contained in the dried reagent such as a metallochromic dye or a pH dependent dye included in the DNA solution would provide an end point indicator for the amplification reaction showing that the target nucleic acid is present (see Example 1 demonstrating that amplification reagents that are rehydrated after freeze drying within a polymer matrix are active and amplify nucleic acid).

Sheets and strips of polymer matrix comprising lyophilized sample(s) within the pores are well suited to these applications. A single sheet may comprise one or all reagents used for a single reaction. Multiple different sheets comprising one or all reagents for one or more reactions may be layered for use in multiple sequential reactions. In such scenarios, the substrate for the reaction(s) in a buffer (and optionally one or more further reagents for the reaction) is added to a first sheet of porous polymer matrix comprising lyophilized reagent(s) within its pores, to cause the lyophilized reagent(s) to become hydrated and to react with the substrate. The reaction may be optionally monitored by a color change. If a second sheet of porous polymer matrix comprising further (e.g. different) reagents lyophilized within its pores is layered or stacked adjacent to the first, the product of the first reaction might diffuse from the first porous polymer matrix into the second porous polymer matrix to hydrate the lyophilized reagents therein and permit a second reaction to occur. An example is shown in FIG. 11D.

Any individual reagent or mixture of reagents may be dried within the matrix as desired for ease of use. Stacking layers of polymer sheets containing different dried (lyophilized) reagents, may be useful in a workflow where an aqueous or oil based liquid containing substrate is added to the top sheet in a stacked layer to hydrate the reagents and the hydrated reagent with substrate diffuses into a second layer allows a sequential reaction to occur. The layers of dried reagent may be interspersed with affinity molecule bound membranes or other membranes that delay or remove certain molecules from layer 1 before the solutions pass into layer 2.

### Lyophilized Sample

The present disclosure also provides a lyophilized sample comprising one or more molecules of interest, contained within the pores of one or more (e.g. one) polymer matrix. In some embodiments, the lyophilized sample is prepared by a method disclosed herein. The lyophilized sample may comprise any one or more molecules of interest as described herein, such as a mastermix containing reagents for a reaction, such as an amplification or reverse transcription reaction. The lyophilized sample may contain one or more excipients, sugars, or buffers as defined herein. The polymer matrix may be any polymer matrix as described herein. In some embodiments, the polymer matrix is in the form of a 3-dimensional block (e.g. cube), cylinder, or sphere (e.g. bead); or the polymer matrix may be substantially flat, such as in the form of a sheet (of any shape e.g. circle or square), strip, or tape. The size, volume, and/or shape of the polymer matrix comprising the lyophilized sample may be determined by the dimensions of a reaction container. The polymer matrix may be positioned on, covered by, or encapsulated within a layer of impermeable material.

In some embodiments, the lyophilized sample is contained within the pores of a single polymer matrix, which may be composed of a single polymer or multiple polymers. The lyophilized sample may be contained within first and second pores of the single polymer matrix; optionally wherein the first pores contain at least a first molecule of interest and the second pores contain at least a second molecule of interest, different from the first molecule of interest.

In other embodiments, the lyophilized sample is contained within the pores of at least a first polymer matrix and a second polymer matrix, different from the first. Optionally, first polymer matrix contains a first molecule of interest within its pores, and the second polymer matrix contains a second molecule of interest within its pores, different from the first molecule of interest. The first and second polymer matrix may be composed of the same or different polymers. Thus, in some embodiments, different molecules of interest are lyophilized within different polymer matrices. The two or more polymer matrices may, for example, be in the form of different sheets, which may be stacked adjacent to each other. The two or more polymer matrices (e.g. sheets) may be separated from each other by an affinity matrix or other separating material.

### Polymer Matrix Comprising Lyophilized Sample

The present disclosure also provides a polymer matrix comprising pores, wherein the pores contain a lyophilized sample comprising one or more molecules of interest. In some embodiments, this polymer matrix is prepared by a method of the disclosure. The lyophilized sample, one or more molecules of interest, and polymer matrix may all be as defined herein. The polymer matrix may comprise any one or more molecules of interest as described herein, such as a mastermix containing reagents for a reaction, such as an amplification or reverse transcription reaction. The polymer matrix may contain one or more excipients, sugars, or buffers as defined herein. The polymer matrix may be any polymer matrix as described herein. In some embodiments, the polymer matrix is in the form of a 3-dimensional block (e.g. cube), cylinder, or sphere (e.g. bead); or the polymer matrix may be substantially flat, such as in the form of a sheet (of any shape e.g. circle or square), strip, or tape. The size, volume, and/or shape of the polymer matrix comprising the lyophilized sample may be determined by the dimensions of a reaction container. The polymer matrix may be positioned on, covered by, or encapsulated within a layer of impermeable material

In some embodiments, the polymer matrix comprises or consists of a single polymer matrix comprising pores, wherein the pores contain the lyophilized sample comprising one or more molecules of interest.

In some embodiments, the polymer matrix comprises first and second pores, which may optionally be of different average size or pore density. In such embodiments, the lyophilized sample may be contained within first and/or second pores of the polymer matrix; for example wherein the first pores contain at least a first molecule of interest and the second pores contain at least a second molecule of interest, different from the first molecule of interest.

In some embodiments, the polymer matrix is composed of two or more different polymers, arranged as at least a first polymer matrix and a second polymer matrix; optionally wherein the pores of the first polymer matrix contain at least a first molecule of interest and the pores of the second polymer matrix contain at least a second molecule of interest, different from the first molecule of interest.

The polymer matrix comprising pores containing the lyophilized sample may be used to store the sample until use, and may therefore be considered to be a sample storage device.

### Reaction Vessel

There is also provided a reaction vessel, comprising one or more lyophilized sample or one or more porous polymer comprising lyophilized sample (sample storage device) as defined herein.

The reaction vessel may take any form in which a reaction may be performed, for example a reaction tube or strip of tubes, or a test device such as a microfluidic device or multi-well dish.

The lyophilized sample, one or more molecules of interest, and polymer matrix may all be as defined herein. The reaction vessel may comprise at least a first lyophilized sample and a second lyophilized sample, or at least a first porous polymer matrix and a second porous polymer matrix; optionally wherein the first porous polymer matrix comprises within the pores a lyophilized sample comprising at least a first molecule of interest or at least a first set molecules of interest and the second porous polymer matrix comprises within the pores a lyophilized sample comprising at least a second molecule of interest or at least a second set of molecules of interest. The first molecule of interest may be the same or different from the second molecule of interest; the first set of molecules of interest may be the same or different from the second set of molecules of interest; or the first set of molecules of interest may contain certain individual molecules of interest that are present in both the first and second porous polymer matrices. For example, the reaction vessel may comprise at least a first porous polymer matrix and a second porous polymer matrix wherein the first porous polymer matrix comprises within the pores a lyophilized sample comprising at least a first molecule of interest and the second porous polymer matrix comprises within the pores a lyophilized sample comprising at least a second molecule of interest, different from the first molecule of interest.

### Kit

The present disclosure further provides a kit comprising: (a) one or more lyophilized sample, polymer matrix (sample storage device), or reaction vessel as defined herein; and (b) aqueous solution for rehydrating the lyophilized sample(s) comprising one or more molecules of interest. The kit may also contain instructions for use.

In one embodiment, a kit is provided that contains: one or more lyophilized samples in a porous polymer strip or tape and optionally on a support strip, in a water impermeable container such as a Mylar bag or cannister; and optionally a reaction vessel (e.g. tube) or test device such as a microfluidic device, 96 well dish or strip of plastic tubes with instructions for use. Optionally a solution for rehydrating the samples may be provided in the kit.

An aqueous solution for rehydration may contain further reagents, enzymes or buffer components to make up the reaction, and/or one or more primers or template molecules. Additional components in a rehydrating solution may be selected according to need, such as surfactants or dispersants to aid release of reagents from the polymer, if required.

The present disclosure further relates to kits including lyophilized materials (e.g., a lyophilized sample in a porous polymer matrix or a porous polymer matrix comprising a lyophilized sample, wherein the lyophilized sample, in each case, comprises one or more molecules of interest). For example, a lyophilized sample may include a molecule interest selected from a polymerase, dNTPs (e.g., one or more NTPs, canonical NTPs, non-canonical NTPs, modified NTPs), primers, other enzymes (e.g., other polymerases, enzymes other than polymerases, or both), buffering agents, or combinations thereof. Enzymes may be included in a storage buffer (e.g., comprising a buffering agent and either comprising glycerol or glycerol-free). A kit may include a reaction buffer which may be in concentrated form, and the buffer may contain additives (e.g. glycerol), salt (e.g. KCl), reducing agent, EDTA or detergents, among others. A kit comprising dNTPs may include one, two, three of all four of dATP, dTTP, dGTP and dCTP. A kit may further comprise one or more modified nucleotides. A kit may optionally comprise one or more primers (random primers, bump primers, exonuclease-resistant primers, chemically-modified primers, custom sequence primers, or combinations thereof).

A kit may be a non-natural collection of components configured, for example, for convenient storage, shipping, delivery, and/or use. One or more components of a kit may be included in one container for a single step reaction, or one or more components may be contained in one container, but separated from other components for sequential use or parallel use. The contents of a kit may be formulated for use in a desired method or process.

A kit is provided that contains: (a)(i) a lyophilized sample in a porous polymer matrix or (ii) a porous polymer matrix comprising a lyophilized sample; and (b) a buffer. A kit may comprise a mastermix, optionally, in combination with, in addition to or in lieu of the buffer. A matermix, if included, may be suitable for receiving and amplifying a template nucleic acid. A molecule of interest may be a purified enzyme so as to contain substantially no DNA or RNA and no nucleases. A buffer may be or comprise a reaction buffer or a storage buffer. A buffer may include a non-ionic surfactant, an ionic surfactant (e.g. an anionic or zwitterionic surfactant) and/or a crowding agent. A kit may include the lyophilized sample and the reaction buffer in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or subpackaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### Rehydration Method

The present disclosure also provides a method, comprising: adding an aqueous solution to one or more lyophilized sample or polymer matrix (e.g. storage device) as defined herein, so as to rehydrate the lyophilized sample comprising one or more molecules of interest. The rehydration method may further comprise releasing or dispersing the rehydrated sample comprising one or more molecules of interest from the pores of the polymer matrix, as described herein.

In some embodiments, the one or more lyophilized sample or porous polymer matrix is in a reaction vessel and the method comprises adding the aqueous solution to the reaction vessel. The one or more rehydrated molecules of interest are released from the pores of the polymer matrix into the reaction vessel and can then be used in a reaction (e.g. upon addition of further reagents, if not already contained in the rehydration solution, and/or upon addition of substrate).

In some embodiments, the reaction vessel comprises at least a first porous polymer matrix and a second porous polymer matrix; wherein the first polymer matrix comprises within the pores a lyophilized sample comprising at least a first molecule of interest and the second polymer matrix comprises within the pores a lyophilized sample comprising at least a second molecule of interest, different from the first molecule of interest; wherein the method comprises adding aqueous solution to the first and second polymer matrices in the reaction vessel, so as to rehydrate the lyophilized samples comprising the first and second molecules of interest. The molecules of interest are released from the porous polymer matrices into the reaction vessel and can then be used in a reaction.

In some embodiments, the method comprises adding to a reaction vessel: (i) aqueous rehydration solution as described herein; (ii) a first lyophilized sample or polymer matrix (e.g. storage device) comprising at least a first molecule of interest, as defined herein, so as to rehydrate the lyophilized sample comprising the first molecule of interest; and (iii) a second lyophilized sample or polymer matrix (e.g. storage device) comprising at least a second molecule of interest, as defined herein, so as to rehydrate the lyophilized sample comprising the second molecule of interest. The first and second lyophilized samples or polymer matrices may be added to the reaction vessel simultaneously with the rehydration solution, to rehydrate the first and second molecules of interest simultaneously for use in the same reaction or same reaction step. Alternatively, the first and second lyophilized samples or polymer matrices may be added to the reaction vessel sequentially, with the same or different rehydration solutions, for use in sequential reactions or sequential steps of the same reaction.

In other embodiments, as discussed above, the porous polymer matrix serves not only as a storage means for a molecule or mixture of molecules of interest but also as a reaction vessel. Thus, an additional reaction vessel is not required. Upon rehydration of the lyophilized sample, the molecule(s) of interest are not released from the polymer matrix but instead remain within the pores or diffuse within the polymer matrix. The molecule(s) of interest can then be used in a reaction (e.g., PCR or LAMP reaction) that proceeds within the polymer matrix. Alternatively, the rehydrated molecule(s) of interest, or the product of a reaction performed using said rehydrated molecules, may diffuse from a first porous polymer matrix to a second porous polymer matrix placed adjacent to the first (e.g. as sheets stacked on top of each other), and then a reaction proceeds within the second polymer matrix. In such embodiments, the rehydration solution may comprise all the additional reagents and/or the substrate (e.g. template nucleic acid) that are required in addition to the rehydrated molecule(s) of interest to complete the reaction. As discussed above, porous polymer matrices in the form of sheets or strips (e.g. placed/stacked adjacent or on top of each other) are particularly well suited to these applications.

### EXAMPLES

### Example 1: Functional Recovery of a qPCR Master Mix after Lyophilization in a Porous Polymer Matrix

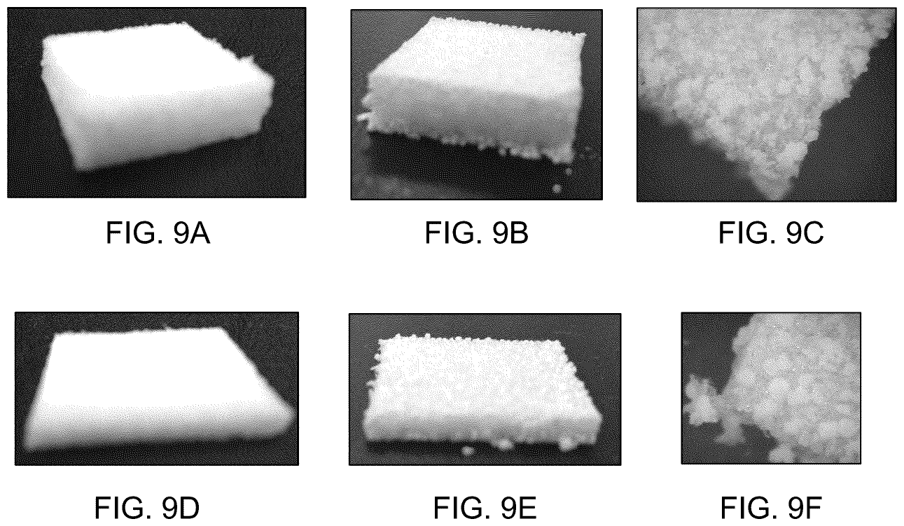

Vyon® PPF and Vyon® F were evaluated to assess their potential use as matrices for qPCR reagents. Samples were evaluated for absorption capability, reagent retention, lyophilization properties (e.g., physical integrity and presence/absence of deformities). FIGS. 9A-9F illustrate examples of untreated Vyon PPF containing lyophilized reagent showing clearly the reagent has been absorbed, retained, successfully lyophilized and has not shown any deformities to the porous polymer matrix. Similar results were obtained with Vyon® F.

Theoretical Absorption volume of hydrophilic Vyon® F cylinders=28.95 μL, Final Volume for 1×=28.95×2=57.9 μL. Average successful extraction percentage=78.6%. Calculated extraction volume from Vyon F cylinders=57.9×0.786=45.51 μL=final reaction volume. Reaction volume excluding template=43.51 μL. Resuspension volume of Vyon F for 43.51 μL extraction volume with no template=43.51/0.786=55.36 μL. 16 Vyon F cylinders were cut at 4.5 mm diameter×4.75 mm thickness. 2 Vials of Cirrus DNA were resuspended with 675 μL for a 2× mix. To a control strip, 22.76 μL Cirrus DNA 2× was added per well. 250 μL of Cirrus DNA 2× was reserved for a non-lyophilized control. In the remaining master mix, the 16 Vyon F cylinders were soaked until saturated (theoretical absorption volume of 28.95 μL). The Vyon F cylinders were placed on a foil lined tray in the freeze-dryer, and the control strip was placed on a multi-rack in the freeze-dryer. Samples were freeze-dried under VirTis® 001-01 (SP Industries, Warminster, PA).

### Post Lyophilization:

A primer probe mix was assembled enough for all wells: 175.18 μL of Beta Actin Forward Primer, Reverse Primer and Probe+273.35 μL water=798.89 μL primer probe mix.

Lyophilized Vyon F samples were placed in extraction strips. To one strip, 55.36 μL of resuspension mix A was added per cylinder. To the other strip of Vyon F, 28.95 μL water added per cylinder for 2× resuspension. Both strips were centrifuged in a plate spinner for 20 seconds for extraction. The strip resuspended to 2× concentration had its extracted contents pooled. Using this master mix: Assembly mix B was made and 43.51 μL was added per well to 4 wells.

The lyophilized control strip was resuspended with 43.51 μL of resuspension mix A. For the non-lyophilized control strip, Assembly mix C was made and had 43.51 μL added per well. To all wells, 2 μL template/NTC added. Wells 1&2—water. Wells 3&4 1×104 Na/rxn. Wells 5&6 1×103 Na/rxn. Wells 7&8 1×102 Na/rxn. The test strip with 4 wells had water in well 1, 1×104 Na/rxn in well 2, 1×103 Na/rxn in well 3 and 1×102 Na/rxn in well 4.

All strips amplified in LC96 as per the protocol in table 2. Resuspension mix A for 20 wells: 471.5 μL PsP+517.2 μL water=988.7 μL. Assembly mix B for 5 wells: 113.8 μL Vyon F extracted 2× master mix+103.75 μL PsP=217.55 μL. Assembly Mix C for 10 wells: 207.5 μL PsP+227.6 μL non-lyophilized 2× master mix=435.1 μL.

TABLE 1
Assay formulation per well

| | | | Volume | |
|---|---|---|---|---|
| | | Volume | Needed per | |
| | | Needed per | Non- | |
| | | Lyophilized | Lyophilized | |
| Reagent | Stoc Conc | well (μl) | well | Final Conc |
| B-Actin Forward | 5 μM | 4.55 | 4.55 | 0.5 μM |
| Primer | | | | |
| B-Actin Reverse | 5 μM | 4.55 | 4.55 | 0.5 μM |
| Primer | | | | |
| B-Actin Probe | 2 μM | 4.55 | 4.55 | 0.2 μM |
| Master Mix | Various | — | 22.76 | Various |
| Water | N/A | 29.86 | 7.1 | N/A |
| Human | 5000 | 2 | 2 | See template |
| Genomic DNA | copies/μL | | | information |

TABLE 2
Thermal profile of amplification

| | | | | | Optical | Optical | | |
|---|---|---|---|---|---|---|---|---|
| | | | | | Excitation | Emission | | |
| | | | | | Channel | Channel | | |
| | | | | Transition | Number | Number | | Cycle |
| Phase | Segment | Temperature | Time | Rate | Name or | Name or | Acquisition | Number |
| Value | (Names) | (*C) | (s) | (*C/s) | value/λnm | value/λnm | Type | Value |
| 1 | HS Activation - | 95 | 60 | 4.4 | N/A | N/A | N/A | 1 |
| | Preincubation | | | | | | | |
| 2 | Denature | 95 | 10 | 4.4 | N/A | N/A | N/A | 45 |
| 3 | Annealing | 60 | 60 | 2.2 | 470 nM | 514 nM | Single | 45 |

Both the lyophilized and non-lyophilized controls were essentially equivalent showing that the reagent can be re-lyophilized successfully with no significant impact on PCR performance.

Successful amplification was seen from both test strips of Vyon F.

### Example 2: Demonstration of the Capability of Successful PCR from a Master Mix Absorbed and Extracted by Vyon Samples

8 Vyon® F cylinder samples were hole-punched with 4.5 mm diameters and 75.55 mm³ volumes.

A complete master mix with primers and probe was formulated and made as per the table below: This mix was scaled and made to be enough for 40 wells to account for ability to submerge the Vyon F samples. 284 per well was pipetted into each well of a control strip.

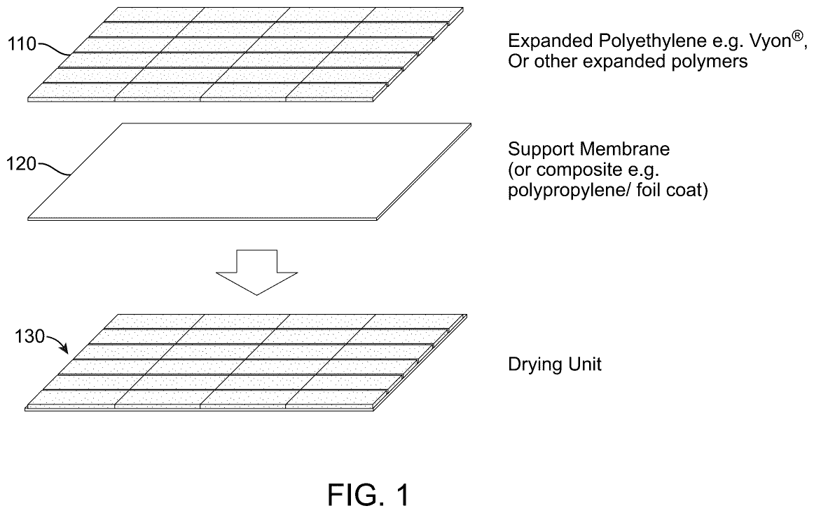

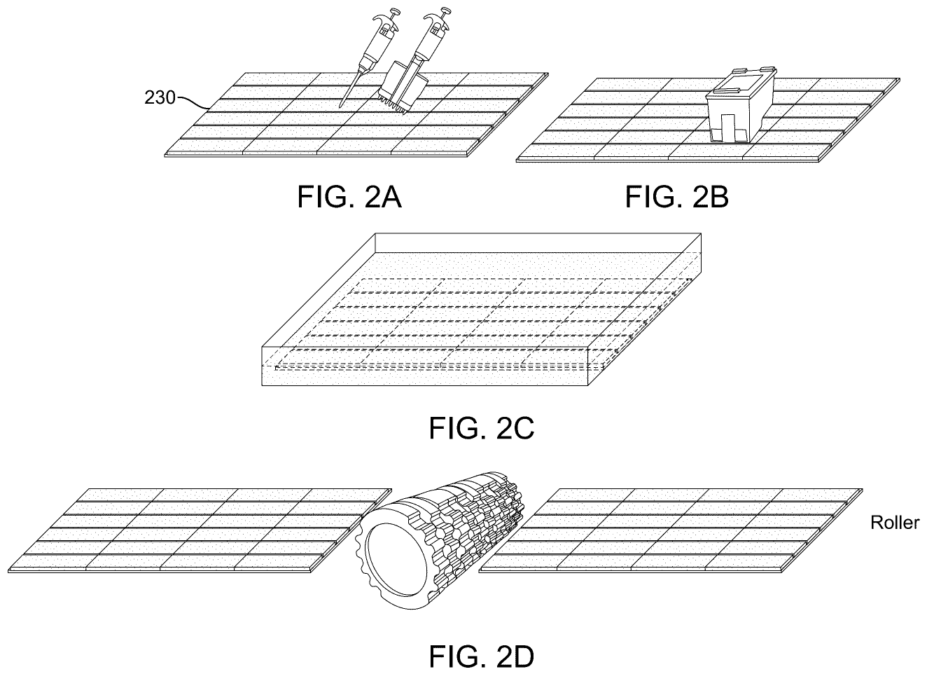

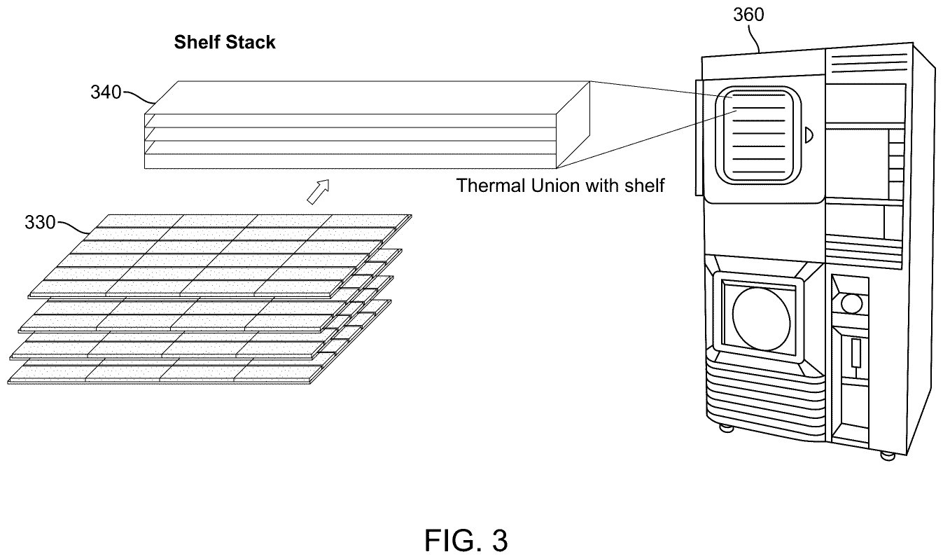

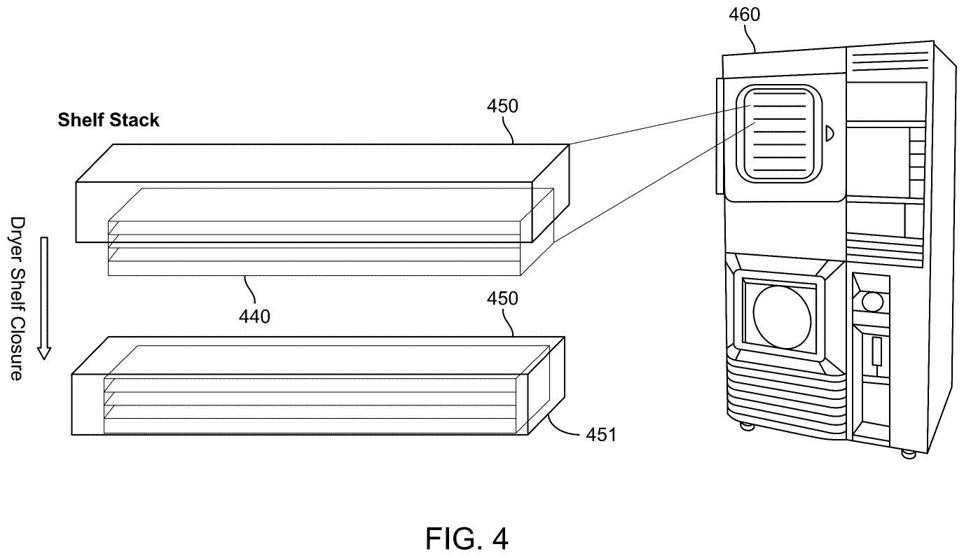

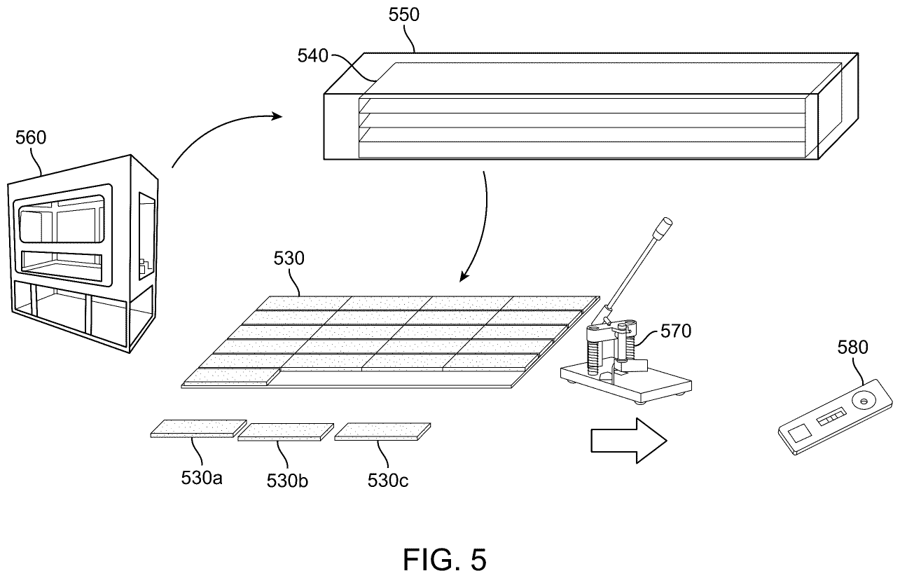

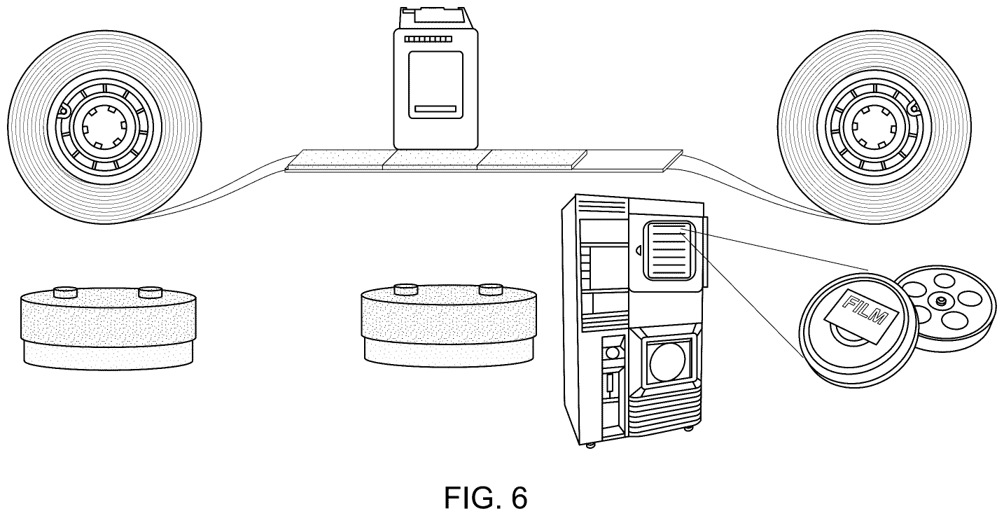

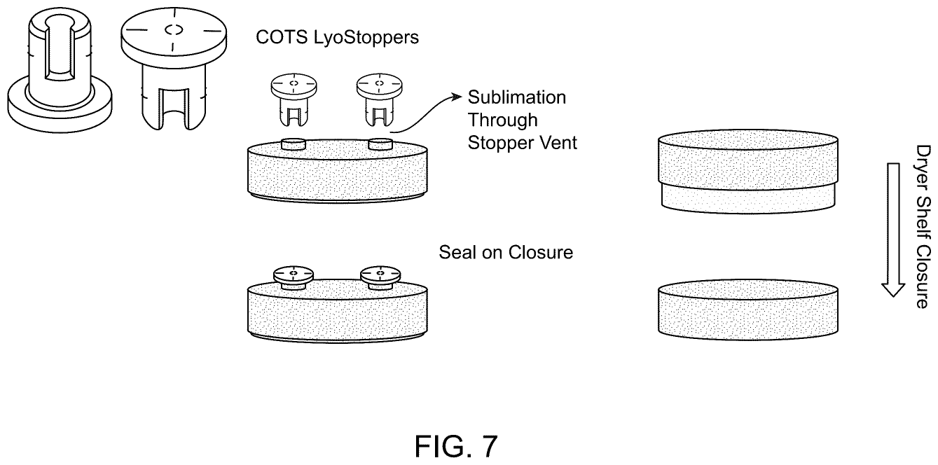

Vyon F cylinders were soaked in the master mix before being placed in the extraction strip device (FIG. 1) and placed on top of the test strip. The strip and device were centrifuged in a plate spinner for ^{˜}20 seconds to extract the master mix from the Vyon F cylinder. Visually, blue reagent was present in the wells showing successful extraction, as well as the now lack of coloring of the Vyon F cylinders.

To all wells of both test and control strip, 2 μL of template/NTC added. Wells 1&2—NTC. Wells 3&4-1×10⁶ Na/rxn. Wells 5&6-1×10⁵ Na/rxn. Wells 7&8-1×10⁴ Na/rxn.

TABLE 3

| | | | | | Optical | Optical | | |
|---|---|---|---|---|---|---|---|---|
| | | | | | Excitation | Emission | | |
| | | | | | Channel | Channel | | |
| | | | | Transition | Number | Number | | Cycle |
| Phase | Segment | Temperature | Time | Rate | Name or | Name or | Acquisition | Number |
| Value | (Names) | (*C) | (s) | (*C/s) | value/λnm | value/λnm | Type | Value |
| 1 | Reverse | 48 | 600 | 4.4 | N/A | N/A | N/A | 1 |
| | Transcriptase | | | | | | | |
| 2 | HS Activation - | 95 | 60 | 4.4 | N/A | N/A | N/A | 1 |
| | Preincubation | | | | | | | |
| 3 | Denature | 95 | 10 | 4.4 | N/A | N/A | N/A | 45 |
| 4 | Annealing | 60 | 60 | 2.2 | 470 nM | 514 nM | Single | 45 |

Results are shown in FIGS. 10A-10D and FIGS. 11A-11B.

As illustrated in FIG. 11A, a qPCR master mix was dosed via saturation into cylinders of porous polymers and then lyophilized. Following lyophilization, master mix was reconstituted via rehydration with an aqueous solution containing primers and probe for target human gene ACTB (β-actin), and then dispensed into reaction tubes by centrifugation. Human genomic DNA template was then added to the reactions followed by amplification, detection and quantitation by real-time qPCR. Reaction conditions: BACT forward and reverse primers at 0.5 μM each; BACT probe at 0.2 μM; human genomic DNA template at 10⁴, 10³ or 10² copies/reaction). Thermocycling conditions: Initial Hot Start activation at 95° C. for 1 minute followed by 45 cycles of denaturation at 95° C. for 10 seconds/annealing and extension at 60° C. for 1 minute. Precursor experiments established dosing parameters for each polymer as summarized in Table 4; this consisted of empirically determining the volume of master mix (i.d) retained within a tested volume of polymer (mm³), thereby providing a polymer property of absorption per unit volume (μl/mm³) polymer property. Together with percent extraction, this provided a means of precise dosing. Amplification curves for real-time detection of fluorescence are shown in FIG. 11B. Top, control reagent (master mix lyophilized in tubes).

TABLE 4

| | | Material | Pore | Liquid | |
|---|---|---|---|---|---|
| | | Thickness | Size | Absorption | Percent |
| Polymer | Material Type | (mm) | (μm) | (μL/mm³) | Extraction |
| Porex ® | Polyethylene | 3.17 | 15-150 | 0.501 | 84.6% |
| Sample A | | | | | |
| Porex ® | Polyethylene | 3.175 | 80-170 | 0.665 | 97.9% |
| Sample B | | | | | |
| Vyon ® F | Polypropylene | 4.75 | 20-40 | 0.383 | 78.6% |
| Sample A | | | | | |

### Example 3: DNA PCR Master Mix can be Lyophilized in Hydrophilic Porex® A, B, C, and D Polymers

3 Vials of Cirrus DNA were resuspended with 675 μL water and pooled for a 2× mix. 8 cylinders of hydrophobic Porex® A, B, C, and D polymers, each with 4.5 mm diameters, were cut using a hole punch. Each set of 8 cylinders was soaked in Cirrus DNA until saturated and placed on a foil-lined metal tray. This was repeated for hydrophilic Vyon F. 200 μL of the Cirrus DNA 2× was reserved for a non-lyophilized control. Into a control strip, 10 μL of Cirrus DNA was added per well. This strip served as a lyophilized control.

Samples were freeze-dried under VirTis 001-01 in an Elmo drier.

### Post Lyophilization:

A primer probe mix was assembled enough for all wells: 270 μL of Beta Actin Forward Primer, Reverse Primer and Probe=800.77 μL primer probe mix. A resuspension mix was made for each sample using the primer probe mix. Lyophilized material samples were placed in extraction strips. To each strip the required volume of resuspension mix (see Table 4) was added per cylinder. These strips were centrifuged in a plate spinner or 20 seconds for extraction.

The lyophilized control strip was resuspended with 18 μL of resuspension mix.
For the non-lyophilized control strip, 18 μL of control mix (100 μL master mix+60 μL PsP+20 μL water) was added per well. To all wells, 2 μL template/NTC added. Wells 1&2—water, Wells 3&4 1×10⁴ Na/rxn, Wells 5&6 1×10³ Na/rxn, Wells 7&8 1×10² Na/rxn. All strips amplified in LC96 as per the protocol in table 3.

Both the lyophilized and non-lyophilized controls amplified successfully. Successful amplification seen from control strip of Vyon F.

TABLE 5
Values and calculations used to determine resuspension volumes required for Porex samples.

| | | | | | | | | Extracted | Resuspension |
|---|---|---|---|---|---|---|---|---|---|
| | | | | | | | | volume | Volume |
| | | | | | | | | required | required to |
| | | | Theoretical | | | Theoretical | Theoretical | to leave | achieve this |
| | | | Resuspension | | | Volume | Volume | 2 μL | extracted volume |
| | | Theoretical | Volume to IX | Percentage | Percentage | extracted | extracted | available | (extracted volume |
| | Cylinder | Absorption | (Double | Extraction | Extraction | from 2X | from IX | for | required/ |
| Porex | Volume | Volume* | absorption | from Lower | from Higher | resuspension | resuspension | template | extraction %) |
| Sample | (mm³) | (μL) | volume) (μL) | volume* | volume* | (μL) | (μL) | (μL) | (μL) |
| A | 50.496 | 25.312 | 50.624 | 84.64% | 65.08% | 21.42 | 32.95 | 30.95 | 47.56 |
| B | 25.248 | 20.9 | 41.8 | 85.8% | 70.65% | 17.93 | 29.53 | 27.53 | 38.97 |
| C | 7.952 | 7.249 | 14.498 | 91.43% | 99.14% | 6.63 | 14.37 | 12.37 | 12.48 |
| D | 50.496 | 33.585 | 67.17 | 97.88% | 65.97% | 32.87 | 44.31 | 42.31 | 64.14 |

*Indicate values which have been obtained experimentally.

TABLE 6
Assay formulation per well.

| Reagent | Stock Conc | Final Conc | Lot. No. |
|---|---|---|---|
| B-Actin Forward Primer | 5 μM | 0.5 μM | MAN1869 |
| B-Actin Reverse Primer | 5 μM | 0.5 μM | MAN1870 |
| B-Actin Probe | 2 μM | 0.2 μM | MAN1871 |
| Master Mix | Various | Various | 20171128A |
| Water | N/A | N/A | RNBK1128 |
| | | | 20210426A |
| Human Genomic DNA | 5000 copies/μL | See template | MAN1823 |
| | | information | |

### Example 4

The experimental aim was to test lyophilised qLAMP master mix, lyophilised on/within a solid phase through resuspension and extraction (for testing reaction outcome). Two different solid phase materials (labelled A & B) were tested. Each of these may be provided in LAMP, and qLAMP (with DNA Binding dye) configurations.

The qLAMP test procedure described below requires the dried material to be extracted from the solid phase amplification (SPA) at the point of reagent dissolution, such that it may then be amplified on a commercial platform.

Alternatively, a user may choose to perform solid phase amplification (SPA) in situ on the solid phase, by direct incubation of the resuspended solid phase in a suitable container (e.g., by sealing/encapsulating the SPA material, such as between layers of plastic) when template is included.

Analysis using processes other than qLAMP may then be applied.

General Instructions:
1. Place Extraction Strip on top of a test strip (see FIGS. 12 and 13).
2. Place SPA cylinders containing lyophilized Master Mix into the extraction strip, 1 cylinder per well. Push the SPA cylinders down gently until they cannot go any lower and are blocking the well. (Insert gently when using material B.)
3. Add resuspension mix—for volume required refer to table below.
4. Place ‘Extraction Strip+Test Strip’ on a plate/rack compatible with a plate spinner, ensure the plate is balanced.
5. Place the plates into a plate spinner and centrifuge for 30 seconds.
6. Remove the plate(s) from the plate spinner and remove the ‘Extraction Strip’ containing the SPA cylinders.
7. Remaining in the test strip are the final reactions without template. Transfer contents to preferred reaction vessels if required.
8. Add 2 μL template/NTC per well, seal the strips, centrifuge, and amplify in a thermal cycler at for 40-60 mins, acquiring on the green channel.

Method Followed:

Two qLAMP master mixes made and lyophilized on different solid phases (cut cylinders of solid phase amplification (SPA) material A and B), and also as free cakes in bioplastics white strips (B59009-1), to be used as lyophilized controls:
RD8074—2× qLAMP Master Mix without Dye
RD8075—2× qLAMP Master Mix with Dye

Post drying, the amplification reagent was recovered after dissolution. To resuspend, SPA samples were inserted into extraction strips placed on top of test strips. The required volume of resuspension mix was added per well (see below). Strips were balanced on plates and centrifuged in a plate spinner for 30 seconds.

TABLE 7
Resuspension mix formulation for RD8074 samples
Volume needed per 1 well reaction (Example Reaction of 25 uL)

| | Stock | Volume | Final |
|---|---|---|---|
| Reagent | Conc | Needed (uL) | Conc |
| MS2 LAMP Primer Mix | 10X | 2.5 | 1X |
| NEB Fluorescent Dye | 20X | 0.625 | 0.5X |
| Water | N/A | 19.875 | N/A |

TABLE 8
Example Resuspension Mix for samples containing dye in the lyophilised material:

| | | | | Volume | | | |
|---|---|---|---|---|---|---|---|
| | | Volume | | Required for | | Concentration | |
| | | Required for | | Resuspension | | in | Concentration |
| | Stock | Resuspension | | Mix For 10 | | Resuspension | in Final |
| Reagent | Concentration | Mix Per Well | | Wells | | Mix | Reaction |
| Water | N/A | 20.5 | μL | 205 | μL | N/A | N/A |
| Primer Mix | 10X | 2.5 | μL | 25 | μL | 1.09X | 1X |

TABLE 9
Resuspension mix formulation for RD8075 samples
Volume needed per 1 well reaction (Example Reaction of 25 uL)

| | Stock | Volume | Final |
|---|---|---|---|
| Reagent | Conc | Needed (uL) | Conc |
| Water | N/A | 20.5 | 0.2 uM |
| MS2 LAMP Primer Mix | 10X | 2.5 | 1X |

The formulations above were scaled up to provide enough resuspension mix for all required wells.

TABLE 10
Volume of resuspension mixes required for each material

| | | | | | Volume of |
|---|---|---|---|---|---|
| | | | | | Resuspension |
| | | | | | Mix Required |
| SPA Material | | | | | Per Well to Achieve |
| (post | | | Reaction Volume | | Relevant Reaction |
| dissolution/ | Reaction | | Excluding | | Volume Excluding |
| recovered) | Volume | | Template | | Template |
| A | 33 | μL | 31 | μL | 47.6 μL |
| B | 25.6 | μL | 23.6 | μL | 23.6 μL |

Extracted reaction mixes were transferred to reaction tubes as necessary. 2 μL MS2 template/water was added per well, reaction tubes were sealed and incubated as per the thermal protocol detailed.

Two separate amplification experiments were completed:
1. RD8074 Samples without dye—comparing master mix extracted directly into wells to master mix extracted, pooled and redistributed at even volumes per well.
2. RD8075 Samples with dye—comparing master mix extracted from SPA lyophilised cylinders to master mix extracted from cylinders cut from lyophilised strips of SPA.

TABLE 11
PCR protocol:

| | | | | | Optical | Optical | | |
|---|---|---|---|---|---|---|---|---|
| | | | | | Excitation | Emission | | |
| | | | | Transition | Channel Number/ | Channel Number/ | | Cycle |
| Phase | Segment | Temperature | Time | Rate | Name or | Name or | Acquisition | Number |
| Value | (Names) | (° C.) | (s) | (° C./s) | (Value/λnm) | (Value/λnm) | Type | Value |
| 1 | Isothermal | 65 | 45 | N/A | 503 nM | 527 nM | Single | 60 |
| 2 | Melting Analysis | 75 | N/A | 0.05 | 503 nM | 527 nM | Continuous | N/A |
| 3 | | 95 | N/A | N/A | | | | |

Template INFORMATION:

MS2 RNA diluted as 1 in 10, 5 μl in 45 μl RNA diluent. Original concentration of MS2 RNA is 5×10⁵ Na/μL. Concentrations used: 5×10⁵ Na/μl, 5×10⁴ Na/μl, 5×10³ Na/μl.
Wells 1&2=NTC
Wells 3&4=5×10⁵ Na/μl (1×10⁶ Na/Rxn)
Wells 5&6=5×10⁴ Na/μl (1×10⁵ Na/Rxn)
Wells 7&8=5×10³ Na/μl (1×10⁴ Na/Rxn)

### Summary Observations (FIGS. 14-16):

SPA Material A and material B both had successful amplification and detection
Material A had more consistency in successful amplification and detection
Material A showed successful detection of amplification when the dye was both incorporated in the lyophilised SPA, and when the dye was added separately post-lyophilisation
Material A showed very similar amplification when comparing individually lyophilised cylinders to cylinders cut from a lyophilised strip
Material A reactions amplified faster than material B reactions
Material B cannot successfully contain and lyophilise the dye, only two wells showed weak amplification.

1-38. (canceled)

39. A method of producing a lyophilized sample, comprising:
lyophilizing a material comprising a porous polymer matrix and a sample solution in pores of the matrix, the sample solution comprising one or more molecules of interest, thereby producing a porous polymer matrix comprising the lyophilized sample.

40. The method of claim 39, further comprising:
contacting the sample solution and the porous polymer matrix to form the material, or
forming the porous polymer matrix comprising the lyophilized sample into one or more pieces having a desired size, volume, and/or shape.

41. The method of claim 39, wherein
the porous polymer matrix has the form of a sheet, a strip, a tape, a block, a cylinder, or a bead, or
the one or more molecules of interest is selected from the group consisting of a protein, a nucleic acid, and one or more nucleoside triphosphates.

42. The method of claim 40, wherein forming the porous polymer matrix further comprises cutting, stamping, punching, tearing or fragmenting the porous polymer matrix comprising the lyophilized sample into one or more pieces having the desired mass, size, volume, and/or shape.

43. The method of claim 40, further comprising:
inserting the piece(s) of polymer matrix into one or more reaction volumes; optionally wherein the piece(s) of polymer matrix comprise(s) a quantity of the one or more molecules of interest that is effective for a reaction in the reaction volume(s); and optionally wherein he reaction volume is in a reaction container or test device.

44. The method of claim 39, wherein the sample solution contains a defined concentration or a predetermined amount of the one or more molecules of interest.

45. The method of claim 39, wherein the sample solution comprises a defined concentration or predetermined amount of the one or more molecules of interest proportioned to produce a defined or predetermined mass to mass or mass to volume ratio of (a)(i) lyophilized sample or (ii) molecule(s) of interest to (b) porous polymer matrix.

46. The method of claim 39, further comprising:
storing the porous polymer matrix containing the lyophilized sample; optionally wherein the stored porous polymer matrix has the form of one or more flat sheets or strips optionally arranged in layers or the form of a tape optionally arranged as a roll, or
after the lyophilizing, sealing the porous polymer matrix containing the lyophilized sample optionally by reversibly bonding the porous polymer matrix to an impermeable membrane or reversibly encapsulating the porous polymer matrix within an impermeable material.

47. The method of claim 39, wherein the sample comprises a DNA polymerase, a reverse transcriptase, and/or one or more primers and/or probes; or wherein the sample is a reagent master mix for DNA amplification or for a reverse transcriptase reaction.

48. The method of claim 39, wherein the sample further comprises one or more excipients and/or sugars in an aqueous buffer.

49. The method of claim 39, wherein the sample comprises one or more non-reducing sugars selected from lactose, sorbitol, mannitol, raffinose, sucrose, trehalose, stachyose, verbascose, isomaltose, cycledextrin, maltodextrin, dextran, branched dextran, and cycloextran; optionally at a concentration in the range of 0.1-20 wt. %.

50. The method of claim 39, wherein the porous polymer matrix comprises or consists of
(a) a sintered synthetic polymer; or
(b) a porous polymer selected from the group consisting of: polyester, polyethylene, polypropylene, polyurethane, polytetrafluoroethylene, polyvinyl, polyvinylidene fluoride, polyvinyl chloride, polyacrylate, polycarbonate, acrylonitrile butadiene styrene, polyamides (such as nylon), polyolefins, cellulose acetate, polylactic acid, polyphenylene sulphide, toluene diisocyanates, methylene diphenyl diisocyanates, hexamethylene diisocyanate; or
(c) a porous polymer selected from the group consisting of fluoropolymers, polyamides, polyethylenes, polypropylenes, polyesters, polyacrylonitriles, polyether imides, polyether ketones, polysulfones, polyethersulfones, polyvinyl chlorides, copolymers of vinyl chloride and acrylonitrile; or
any combination thereof.

51. A lyophilized sample comprising one or more molecules of interest, contained within pores of one or more porous polymer matrices.

52. The lyophilized sample of claim 51, wherein
the porous polymer matrix has the form of a film, a sheet, a strip, a tape, a block, a cylinder, or a bead, or
the one or more molecules of interest is selected from the group consisting of a protein, a nucleic acid, and one or more nucleoside triphosphates, or
the lyophilized sample is contained within first and second pores of the polymer matrix; optionally wherein the first pores contain at least a first molecule of interest and the second pores contain at least a second molecule of interest, different from the first molecule of interest, or
the lyophilized sample is contained within the pores of at least a first polymer matrix and a second polymer matrix; optionally wherein the pores of the first polymer matrix contain at least a first molecule of interest and the pores of the second polymer matrix contain at least a second molecule of interest, different from the first molecule of interest.

53. The lyophilized sample of claim 51, wherein the first polymer matrix and the second polymer matrix are in the form of first and second sheets of polymer matrix, optionally wherein the first and second sheets of polymer matrix are adjacent to each other, optionally separated by an affinity matrix or other separating material.

54. The lyophilized sample according to claim 51, wherein the polymer matrix is positioned on or encapsulated within a layer of impermeable material.

55. The lyophilized sample of claim 51, wherein the sample comprises a DNA polymerase, a reverse transcriptase, and/or one or more primers and/or probes; or wherein the sample is a reagent master mix for DNA amplification or for a reverse transcriptase reaction.

56. The lyophilized sample of claim 51, wherein the sample further comprises one or more excipients and/or sugars in an aqueous buffer.

57. The method, lyophilized sample, or porous polymer matrix of claim 51, wherein the sample comprises one or more non-reducing sugars selected from lactose, sorbitol, mannitol, raffinose, sucrose, trehalose, stachyose, verbascose, isomaltose, cycledextrin, maltodextrin, dextran, branched dextran, and cycloextran; optionally at a concentration in the range of 0.1-20 wt. %.

58. The lyophilized sample of claim 51, wherein the porous polymer matrix comprises or consists of
(a) a sintered synthetic polymer; or
(b) a porous polymer selected from the group consisting of: polyester, polyethylene, polypropylene, polyurethane, polytetrafluoroethylene, polyvinyl, polyvinylidene fluoride, polyvinyl chloride, polyacrylate, polycarbonate, acrylonitrile butadiene styrene, polyamides (such as nylon), polyolefins, cellulose acetate, polylactic acid, polyphenylene sulphide, toluene diisocyanates, methylene diphenyl diisocyanates, hexamethylene diisocyanate; or
(c) a porous polymer selected from the group consisting of fluoropolymers, polyamides, polyethylenes, polypropylenes, polyesters, polyacrylonitriles, polyether imides, polyether ketones, polysulfones, polyethersulfones, polyvinyl chlorides, copolymers of vinyl chloride and acrylonitrile; or
any combination thereof.

59. A porous polymer matrix containing within its pores a lyophilized sample comprising one or more molecules of interest.

60. The porous polymer matrix of claim 59, wherein
the polymer matrix is in the form of a film, a sheet, a strip, a tape, a block, a cylinder, or a bead, or
the one or more molecules of interest is selected from the group consisting of a protein, a nucleic acid, and one or more nucleoside triphosphates, or
the lyophilized sample is contained within first and second pores of the polymer matrix; optionally wherein the first pores contain at least a first molecule of interest and the second pores contain at least a second molecule of interest, different from the first molecule of interest, or
the polymer matrix is positioned on or encapsulated within a layer of impermeable material.

61. The porous polymer matrix of claim 59, wherein the sample comprises two or more molecules of interest selected from the group consisting of a protein, a nucleic acid, and one or more nucleoside triphosphates.

62. The porous polymer matrix of claim 59, wherein the sample comprises a DNA polymerase, a reverse transcriptase, and/or one or more primers and/or probes; or wherein the sample is a reagent master mix for DNA amplification or for a reverse transcriptase reaction.

63. The porous polymer matrix of claim 59, wherein the sample further comprises one or more excipients and/or sugars in an aqueous buffer.

64. The porous polymer matrix of claim 59, wherein the sample comprises one or more non-reducing sugars selected from lactose, sorbitol, mannitol, raffinose, sucrose, trehalose, stachyose, verbascose, isomaltose, cycledextrin, maltodextrin, dextran, branched dextran, and cycloextran; optionally at a concentration in the range of 0.1-20 wt. %.

65. The porous polymer matrix of claim 59, wherein the porous polymer matrix comprises or consists of
(a) a sintered synthetic polymer; or
(b) a porous polymer selected from the group consisting of: polyester, polyethylene, polypropylene, polyurethane, polytetrafluoroethylene, polyvinyl, polyvinylidene fluoride, polyvinyl chloride, polyacrylate, polycarbonate, acrylonitrile butadiene styrene, polyamides (such as nylon), polyolefins, cellulose acetate, polylactic acid, polyphenylene sulphide, toluene diisocyanates, methylene diphenyl diisocyanates, hexamethylene diisocyanate; or
(c) a porous polymer selected from the group consisting of fluoropolymers, polyamides, polyethylenes, polypropylenes, polyesters, polyacrylonitriles, polyether imides, polyether ketones, polysulfones, polyethersulfones, polyvinyl chlorides, copolymers of vinyl chloride and acrylonitrile; or
any combination thereof.

66. A method, comprising:
adding an aqueous solution to one or more lyophilized samples of claim 51, to produce a hydrated lyophilized sample comprising one or more molecules of interest.

67. A method, comprising:
adding an aqueous solution to one or more porous polymer matrices of claim 59, to produce a hydrated lyophilized sample comprising one or more molecules of interest.

68. The method of claim 67, further comprising dispersing the rehydrated sample comprising one or more molecules of interest from the one or more porous polymer matrix.
